(19) United States
(12) Patent Application Publication
(10) Pub. No.: US 2014/0323757 A1
(43) Pub. Date: Oct. 30, 2014

(54) Biosurfactant Isolated from Yeast

(71) Applicant: Gyeongbuk Institute of Marine Bio-industry, Gyeongsanbuk-do (KR)

(72) Inventor: Jong Shik Kim, Gyeongsangbuk-do (KR)

(21) Appl. No.: 13/941,983

(22) Filed: Jul. 15, 2013

(30) Foreign Application Priority Data
Apr. 30, 2013 (KR) 10-2013-0047985

(51) Int. Cl. C07C 69/33; A61K 8/37; C11D 1/66; C12R 1/645; C07C 67/48

(52) U.S. Cl. CPC C07C 69/33; C12R 1/645; C07C 67/48; C11D 1/667; A61K 8/375
USPC 560/248; 435/255.1; 560/231

(57) ABSTRACT

The present invention provides a novel compound isolated from yeasts and its use as a bio-surfactant. The bio-surfactant of the present invention shows high surfactant activity, is biodegradable, and is safe to the human body due to its low toxicity. Also, the bio-surfactant of the present invention can be eco-friendly produced through a cultivation of a microorganism in large quantities.

### CROSS-REFERENCE TO RELATED APPLICATION

This application claims under 35 U.S.C. §119(a) the benefit of Korean Patent Application No. 10-2013-0047985 filed Apr. 30, 2013, the entire contents of which are incorporated herein by reference.

### BACKGROUND

(a) Technical Field

The present disclosure relates to new compounds isolated from yeasts and a use thereof as a surfactant.

(b) Background Art

Surfactants are widely used in various industrial fields including medicines, agriculture, cosmetics and the like. Most surfactants currently used in industries are synthetic products chemically made from petroleum, and more than about ten million surfactants have been manufactured worldwide via chemical synthesis. However, due to the growing public concerns over environmental pollution and consumers' preference towards eco-friendly products, there is an urgent need for the development of eco-friendly surfactants which can replace chemically synthesized surfactants.

Further, the progression in biotechnology suggests the use of bio-surfactants (biological surfactants) derived from microorganisms. Bio-surfactants are biodegradable surfactants with an amphiphilic property that are produced by a microorganism. The bio-surfactants are advantageous over the chemically synthesized surfactants in that they are biodegradable, active at extreme temperature or pH conditions, and demonstrate relatively low toxicity. Since the microorganism-derived bio-surfactants have eco-friendly characteristics and can be produced in large scale via fermentation, they can be applied to various fields including oil recovery, medicines, foods and cosmetics, percutaneous drug delivery system (DDS), etc., depending on their intended uses, and thus studies have been actively focused thereon. Until recently, various microorganism-derived surfactants have been used, but so far they have not been shown advantageous due to their relatively low surfactant activity. Thus, there is still a need for the development of bio-surfactants with much stronger activity.

Accordingly, the inventors of the present invention have made numerous efforts to develop a novel compound having strong surfactant activity, which is derived from a natural substance, and safe to the human body. As a result, the present inventors have finally discovered that novel compounds isolated from certain species of yeast have superior surfactant activity.

### SUMMARY OF THE DISCLOSURE

The present invention has been made in an effort to solve the above-described problems associated with prior art.

In one aspect, the present invention provides a compound represented by the following Formula 1:

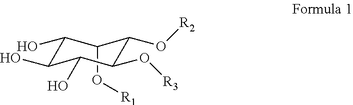

wherein R₁ to R₃ are independently hydrogen or —COR₄;

R₄ is C₁-C₁₀ alkyl; and

at least one of R₁ to R₃ should be —COC₅H₁₁.

In an exemplary embodiment, the compound is represented by Formula 1 wherein R₄ is C₁-C₈ alkyl.

In another exemplary embodiment, the compound is represented by Formula 1 wherein R₄ is C₁-C₅ alkyl.

In still another exemplary embodiment, the compound is represented by Formula 1 wherein R₄ is methyl or pentyl.

In yet another exemplary embodiment, the compound is selected from the group consisting of compounds represented by the following Formulae 2 to 6:

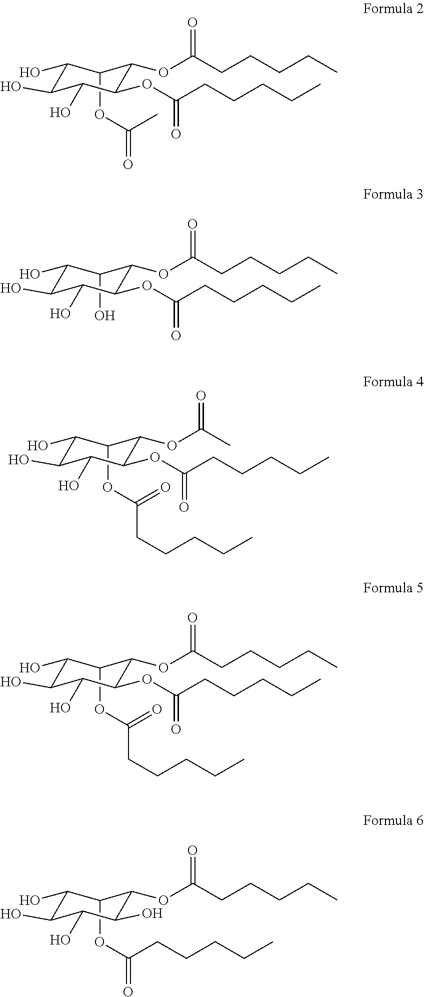 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

In still yet another exemplary embodiment, the compound is isolated from a yeast strain.

In a further exemplary embodiment, the yeast strain is an Aureobasidium sp. strain deposited with Accession No. KCCM11373P.

In another further exemplary embodiment, the compound is a bio-surfactant.

In another aspect, the present invention provides a cleaning composition including the compounds as described above.

In still another aspect, the present invention provides a cosmetic composition including the compounds as described above.

In a further aspect, the present invention provides a method for preparing a bio-surfactant, including:

isolating a compound selected from one or more of the compounds represented by the following Formulae 2 to 6 from an Aureobasidium sp. strain deposited with Accession No. KCCM11373P:

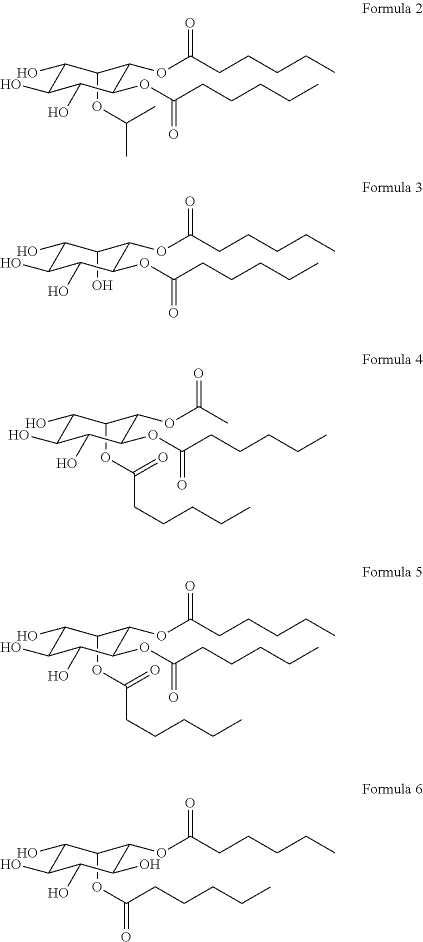 [SMILES: CCCCCC(=O)OC1C(O)C(O)C(O)C(OC(C)C)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

In yet another aspect, the present invention provides an Aureobasidium sp. strain deposited with Accession No. KCCM11373P which produces a compound selected from one or more of the compounds represented by the following Formulae 2 to 6:

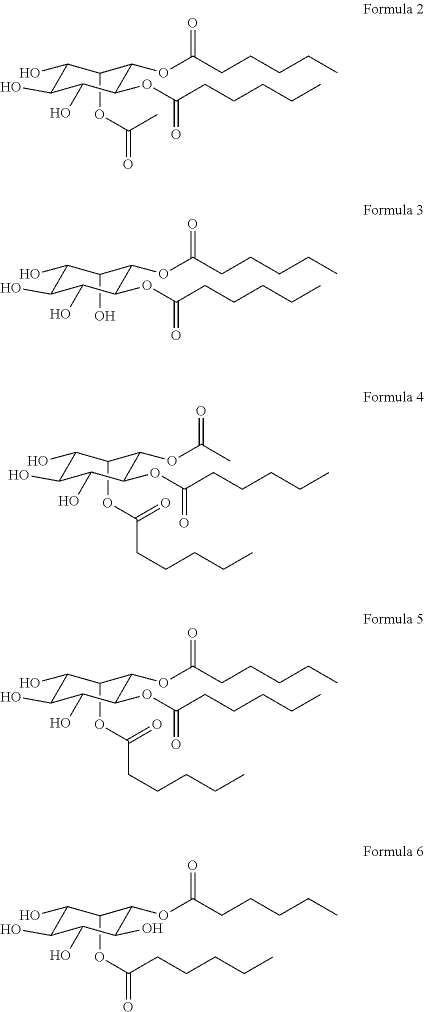 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

Other aspects and preferred embodiments of the invention, as well as other features of the invention are discussed herein.

### BRIEF DESCRIPTION OF THE DRAWINGS

The above disclosure and other features of the present invention will now be described in detail with reference to certain exemplary embodiments thereof, and illustrated in the accompanying drawings which are provided below by way of illustration only, and thus do not limit the present invention:

FIG. 1 is a diagram schematically illustrating the isolation and purification of an active compound.

FIG. 2 is a ¹H NMR spectrum of compound A57-4-gly-1.

FIG. 3 is a ¹³C NMR spectrum of compound A57-4-gly-1.

FIG. 4 is a ¹H-¹H COSY spectrum of compound A57-4-gly-1.

FIG. 5 is a partial structure of compound A57-4-gly-1 identified with a ¹H-¹H COSY spectrum.

FIG. 6 is a HMQC spectrum of compound A57-4-gly-1.

FIG. 7 is a HMBC spectrum of compound A57-4-gly-1.

FIG. 8 is a chemical structure of compound A57-4-gly-1 identified with a 2-Dimensional NMR spectrum.

FIG. 9 is a NMR assignment of proton and carbon peaks of compound A57-4-gly-1.

FIG. 10 is an ESI-mass spectrum of compound A57-4-gly-1.

FIG. 11 is a ¹H NMR spectrum of compound A57-4-gly-2.

FIG. 12 is a ¹³C NMR spectrum of compound A57-4-gly-2.

FIG. 13 is a ¹H-¹H COSY spectrum of compound A57-4-gly-2.

FIG. 14 is a partial structure of compound A57-4-gly-2 identified with a ¹H-¹H COSY spectrum.

FIG. 15 is a HMQC spectrum of compound A57-4-gly-2.

FIG. 16 is a HMBC spectrum of compound A57-4-gly-2.

FIG. 17 is a chemical structure of compound A57-4-gly-2 identified with a 2-Dimensional NMR spectrum.

FIG. 18 is a NMR assignment of proton and carbon peaks of compound A57-4-gly-2.

FIG. 19 is an ESI-mass spectrum of compound A57-4-gly-2.

FIG. 20 is a ¹H NMR spectrum of compound A57-4-gly-3.

FIG. 21 is a ¹³C NMR spectrum of compound A57-4-gly-3.

FIG. 22 is a ¹H-¹H COSY spectrum of compound A57-4-gly-3.

FIG. 23 is a partial structure of compound A57-4-gly-3 identified with a ¹H-¹H COSY spectrum.

FIG. 24 is a HMQC spectrum of compound A57-4-gly-3.

FIG. 25 is a HMBC spectrum of compound A57-4-gly-3.

FIG. 26 is a chemical structure of compound A57-4-gly-3 identified with a 2-Dimensional NMR spectrum.

FIG. 27 is a NMR assignment of proton and carbon peaks of compound A57-4-gly-3.

FIG. 28 is an ESI-mass spectrum of compound A57-4-gly-3.

FIG. 29 is a ¹H NMR spectrum of compound A57-4-gly-4.

FIG. 30 is a ¹³C NMR spectrum of compound A57-4-gly-4.

FIG. 31 is a ¹H-¹H COSY spectrum of compound A57-4-gly-4.

FIG. 32 is a partial structure of compound A57-4-gly-4 identified with a ¹H-¹H COSY spectrum.

FIG. 33 is a HMQC spectrum of compound A57-4-gly-4.

FIG. 34 is a HMBC spectrum of compound A57-4-gly-4.

FIG. 35 is a chemical structure of compound A57-4-gly-4 identified with a 2-Dimensional NMR spectrum.

FIG. 36 is a NMR assignment of proton and carbon peaks of compound A57-4-gly-4.

FIG. 37 is an ESI-mass spectrum of compound A57-4-gly-4.

FIG. 38 is a ¹H NMR spectrum of compound A57-4-gly-5.

FIG. 39 is a ¹³C NMR spectrum of compound A57-4-gly-5.

FIG. 40 is a ¹H-¹H COSY spectrum of compound A57-4-gly-5.

FIG. 41 is a partial structure of compound A57-4-gly-5 identified with a ¹H-¹H COSY spectrum.

FIG. 42 is a HMQC spectrum of compound A57-4-gly-5.

FIG. 43 is a HMBC spectrum of compound A57-4-gly-5.

FIG. 44 is a chemical structure of compound A57-4-gly-5 identified with a 2-Dimensional NMR spectrum.

FIG. 45 is a NMR assignment of proton and carbon peaks of compound A57-4-gly-5.

FIG. 46 is an ESI-mass spectrum of compound A57-4-gly-5.

FIG. 47 is a chemical structure of an active ingredient isolated from a yeast strain.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use.

In the figures, certain reference numbers refer to the same or equivalent parts of the present invention in several figures of the drawings.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention provides a compound represented by the following Formula 1:

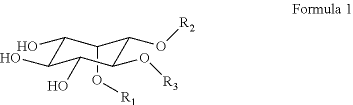

wherein R₁ to R₃ are independently hydrogen or —COR₄;

R₄ is C₁-C₁₀ alkyl; and at least one of R₁ to R₃ should be —COC₅H₁₁.

The term “compound” as used herein is intended to include compounds represented by Formulae 1 to 6, isomers and salts thereof, and should not be construed as that it is restricted to the compounds of Formulae 1 to 6.

In the compound of Formula 1, R₁ to R₃ are independently hydrogen or —COR₄, and R₄ is C₁-C₁₀ alkyl, preferably C₁-C₈ alkyl, more preferably C₁-C₅ alkyl, most preferably methyl or pentyl.

In another exemplary embodiment, the compound of the present invention is a compound selected from the group consisting of following Formula 2 to 6:

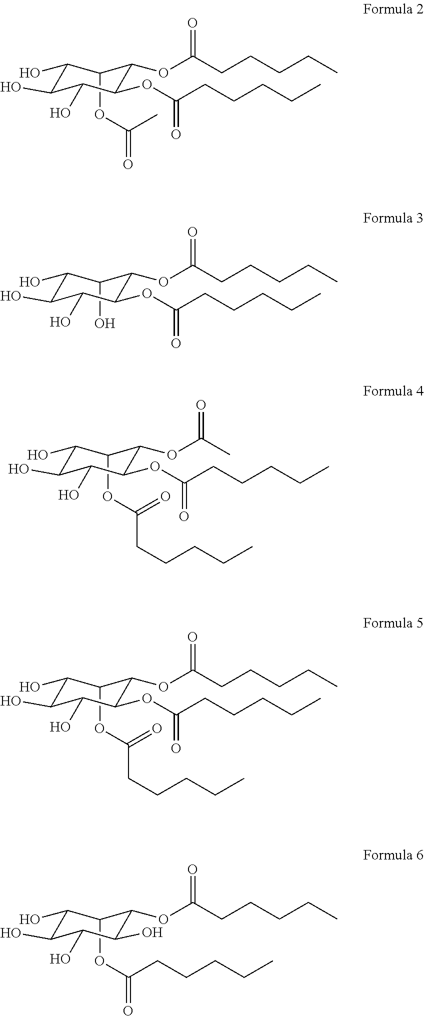 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

In still another exemplary embodiment, the compound of the present invention is isolated from a yeast strain.

The yeast strain suitable for the present invention may include various yeast strains well-known in the art, and in an embodiment the yeast strain is an Aureobasidium sp. strain deposited with Accession No. KCCM11373P.

In yet another exemplary embodiment, the compound of the present invention is characterized by showing bio-surfactant activity.

As compared with conventional synthetic surfactants, the bio-surfactant shows low toxicity and high biodegradability, which allows it to overcome environmental pollution problems raised in the prior art. In addition, since the bio-surfactant has a complicated chemical structure which cannot be easily synthesized by conventional methods, it can be used for a special purpose. Further, the bio-surfactant exhibits equal or similar physiochemical properties, including ability of reducing surface tension and stability to temperature and pH to conventional chemically synthesized surfactants, and thus, it is very useful (Ishigami et al., 1987. Chem. Lett., 763).

The compound of the present invention shows considerably low surface tension, suggesting that it possesses high surfactant activity. The compound of the present invention preferably has a surface tension of 10 to 40 N/m, more preferably 20 to 30 N/m, and most preferably 22 to 29 N/m. When compared with surface tension of conventional surfactants (e.g. 43 N/m of Trehalose lipid and Iturin; 35 N/m of Sophorolipid; 31.4 N/m of Rhamnolipid), the compound of the present invention shows superior surface tension compared to conventional surfactants.

In another aspect, the present invention provides a cleaning composition including the above compound.

The compound suitable to use as a bio-surfactant according to the present invention exhibits strong surfactant activity, and in particular is ideally suited to the field of fabric washing and cleaning. In addition, the bio-surfactant of the present invention can be used in cleaning and polishing hard surfaces.

In still another aspect, the present invention provides a cosmetic composition including the above compound.

The compound of the present invention can be used as a bio-surfactant, and thus it can be advantageously used as an emulsifying agent in the manufacture of soaps, shampoos, creams or lotions.

Besides the above uses, the compound of the present invention can be applied to most industrial fields where a chemically synthesized surfactant has been widely used, for example and without limitation, medicines, foods, secondary oil recovery, pulp and paper industry, purification of oil-contaminated soil and sea water, degradation of milk fat in a bioreactor and the like.

In a further aspect, the present invention provides a method for preparing a bio-surfactant, including: isolating a compound selected from the compounds represented by the following Formulae 2 to 6 from an Aureobasidium sp. strain deposited with Accession No. KCCM11373P:

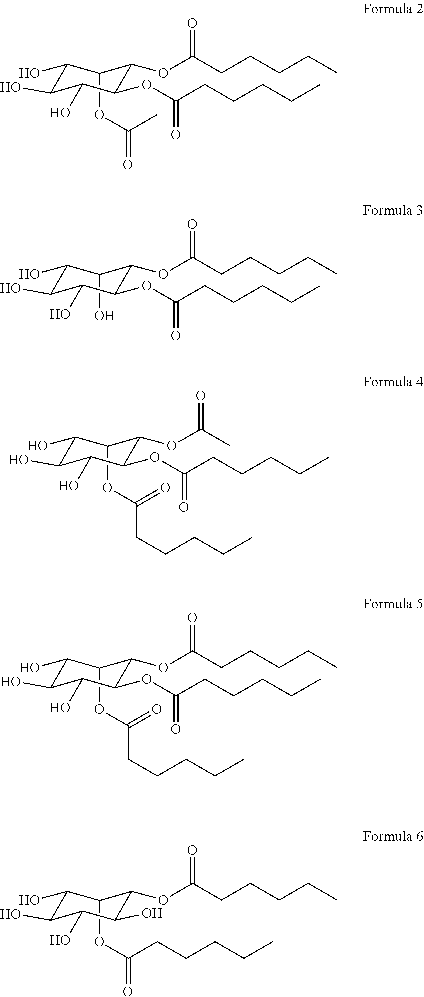 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

In yet another aspect, the present invention provides an Aureobasidium sp. strain deposited with Accession No. KCCM11373P which produces a compound selected from ones represented by above Formulae 2 to 6:

Features and advantages of the present invention are summarized below:
(i) The present invention provides a novel compound isolated from a yeast strain.
(ii) The present invention provides the use of the compound as a bio-surfactant.
(iii) The bio-surfactant of the present invention shows strong surfactant activity, is biodegradable, has a relatively low toxicity, and thus is safe to the human body.
(iv) The bio-surfactant of the present invention can be mass-produced by cultivation of a microorganism, which is eco-friendly.

### EXAMPLES

The following examples illustrate the invention and are not intended to limit the same.

Experimental Materials and Methods

1. Yeast

A yeast fermented material (about 25 L) as a public material for study was obtained from Gyeongbuk Institute for Marine Bio-Industry in a freeze-dried state. The yeast was an Aureobasidium sp. strain which was deposited at Korean Culture Center of Microorganisms (KCCM) on Feb. 7, 2013 (Accession No. KCCM11373P).

2. Analyses

Measurement of Mass Spectrum

FAB-mass spectra were measured on a Jeol JMS-700 MSTATION mass spectrometer (Japan) using glycerol or m-nitrobenzyl alcohol as a matrix. For high-resolution FAB-mass, polyethylene glycol was used as an internal standard.

Measurement of NMR Spectrum

NMR spectra were measured on a Jeol JNM-ECA600 600 MHz FT-NMR spectrometer (Japan) using TMS (tetramethylsilane) as an internal standard. Here, CDCl₃ was used as a solvent, and a chemical shift was expressed in ppm (δ). For NMR spectra, two-dimensional NMR such as ¹H-¹H COSY, HMQC or HMBC as well as one-dimensional NMR such as ¹H NMR or ¹³C NMR were employed.

Reagents

Solvents including hexane, ethyl acetate, chloroform, methanol and acetone used in each purifying step and column chromatography were purchased from SK Chemicals Co., Ltd. (Korea) and Daejung Chemical & Materials Co., Ltd. (Korea). HPLC solvents were purchased from Merck (Germany) and Baxter (Burdick & Jackson, USA), and NMR solvents such as CDCl₃ were purchased from Aldrich (USA). For the isolation and purification of a material, normal-phase TLC (Merck, Kieselgel 60F, 70-230 mesh, USA) and reverse-phase TLC (Merck, RP-18, F₂₅₄, USA), Sephadex LH-20 (Pharmacia, bead size 25-100 μm, Sweden), ODS sep-pak cartridge (Alltech, RP-18, USA) were employed.

Activity Measurement

Surfactant activity was determined by dissolving a compound in water, loading 50 μL of the resulting solution on a parafilm and measuring the degree of spreading thereof. The degree of spreading of a compound was represented by diameter. At this time, an equal amount of distilled water was used as a control.

Experimental Results

1. Isolation, Purification and Surfactant Activity of an Active Ingredient

After a freeze-dried culture solution (about 25 L), provided from the Gyeongbuk Institute for Marine Bio-Industry, was dissolved in water, the resulting solution was subjected to partition extraction with hexane so as to remove a lipid fraction, and this step was followed by partition extraction with ethyl acetate (18 L) twice. The resulting ethyl acetate layer, having an activity as shown below, was dried over magnesium sulfate anhydrous, concentrated under reduced pressure, and purified with flash normal phase (silica gel) column chromatography using chloroform:methanol (50:1→2:1, v/v) as an eluting solvent.

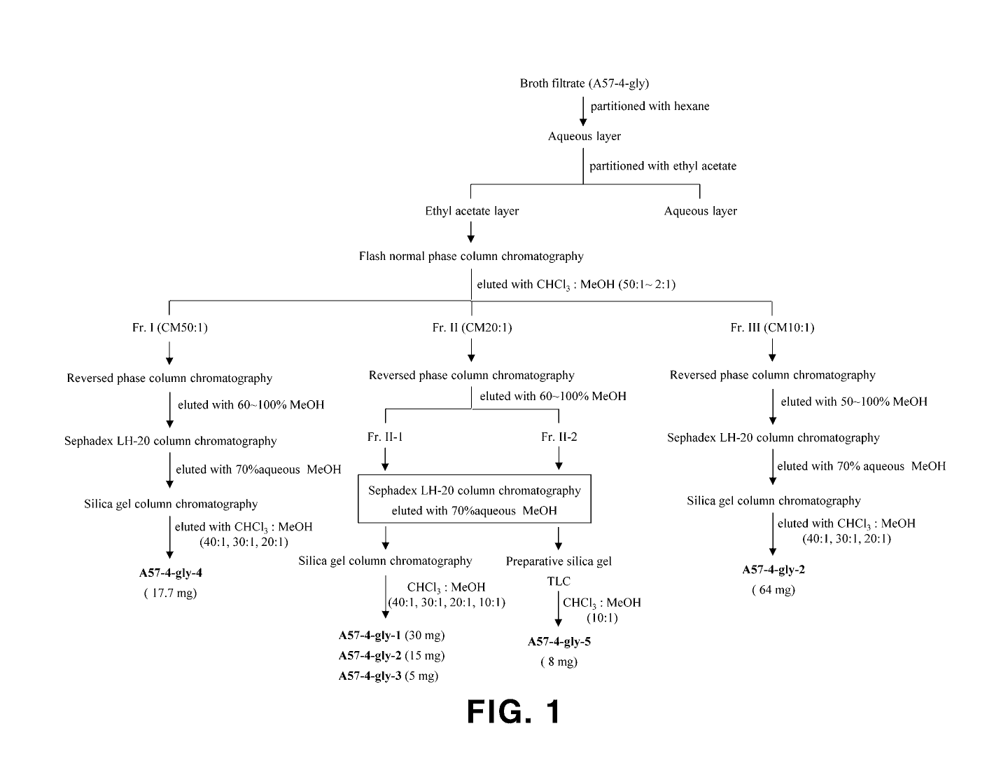

As a result, two fractions of CHCl₃:MeOH(50:1) (Fr. I) and CHCl₃:MeOH(20:1) (Fr. II) showed the highest surfactant activity. In addition, the fraction of CHCl₃:MeOH(10:1) (Fr. III) showed relatively lower surfactant activity than the above two fractions, but it contained an abundance of material. Therefore, the above three fractions were selected, isolated and purified (FIG. 1).

(1) Isolation and Purification of Compounds A57-4-gly-1, A57-4-gly-2 and A57-4-gly-3

Active fraction Fr. II was dissolved in 60% methanol and purified with reversed-phase column chromatography. Here, elution was performed with gradually increasing concentrations of methanol (60%→100%).

As a result, Fr. II was divided into two active fractions of CM20:-RP4-7 (Fr. II-1) and CM20:-RP16 (Fr. II-2). Fr. II-1 was purified with Sephadex LH-20 column chromatography using 70% methanol as an eluting solvent, followed by silica gel column chromatography using chloroform:methanol (40:1→20:1, v/v) as an eluting solvent. Each fraction was analyzed with silica gel TLC using chloroform:methanol (10:1). Since an active compound did not show UV adsorption, a cerium molybdate reagent (10 g of cerium sulfate, 25 g of ammonium hepamolybdate, 100 ml of sulfuric acid, 900 ml of water) was sprayed thereon to develop a color.

As a result, compounds A57-4-gly-1(frs. 23-32, 14.2 mg), A57-4-gly-2(frs. 48-61, 7.3 mg), and A57-4-gly-3(frs. 14-18, 2.5 mg) were purified.

In addition, the active fraction Fr. III was dissolved in 50% methanol and purified with reversed-phase column chromatography. Here, elution was performed with gradually increasing concentrations of methanol (50%→100%).

After concentration of active fractions CM10:1 RP-10-13, it was purified with Sephadex LH-20 column chromatography using 70% methanol, followed by silica gel column chromatography using chloroform:methanol (40:1→20:1, v/v) as an eluting solvent, to thereby further purify A57-4-gly-2 (64 mg).

(2) Isolation and Purification of Compound A57-4-gly-4

The active fraction Fr. I was dissolved in 60% methanol and purified with reversed-phase column chromatography. Here, elution was performed with gradually increasing concentrations of methanol (60%→100%).

After concentration of active fractions CM50:1 RP-4˜9, it was purified with Sephadex LH-20 column chromatography using 70% methanol, followed by silica gel column chromatography using chloroform:methanol (40:1→20:1, v/v) as an eluting solvent, to thereby further purify A57-4-gly-4 (17.7 mg).

(3) Isolation and Purification of Compound A57-4-gly-5

The active fraction Fr. II-2, which was derived from the active fraction Fr. II, was purified with Sephadex LH-20 column chromatography using 70% methanol, and then the active fraction 26 was purified with preparative silica gel TLC using chloroform:methanol (10:1) as a developing solvent, to thereby purify compound A57-4-gly-5 (8 mg) which was adjacent to Rf value of 0.3.

2. Chemical Structure of Active Ingredient

(1) Compound A57-4-gly-1

In order to investigate a chemical structure of compound A57-4-gly-1, It was dissolved in CDCl₃, and subjected to ¹H NMR, ¹³C NMR, ¹H-¹H COSY, HMQC and HMBC analyses.

Measurement and Interpretation of a ¹H NMR Spectrum:

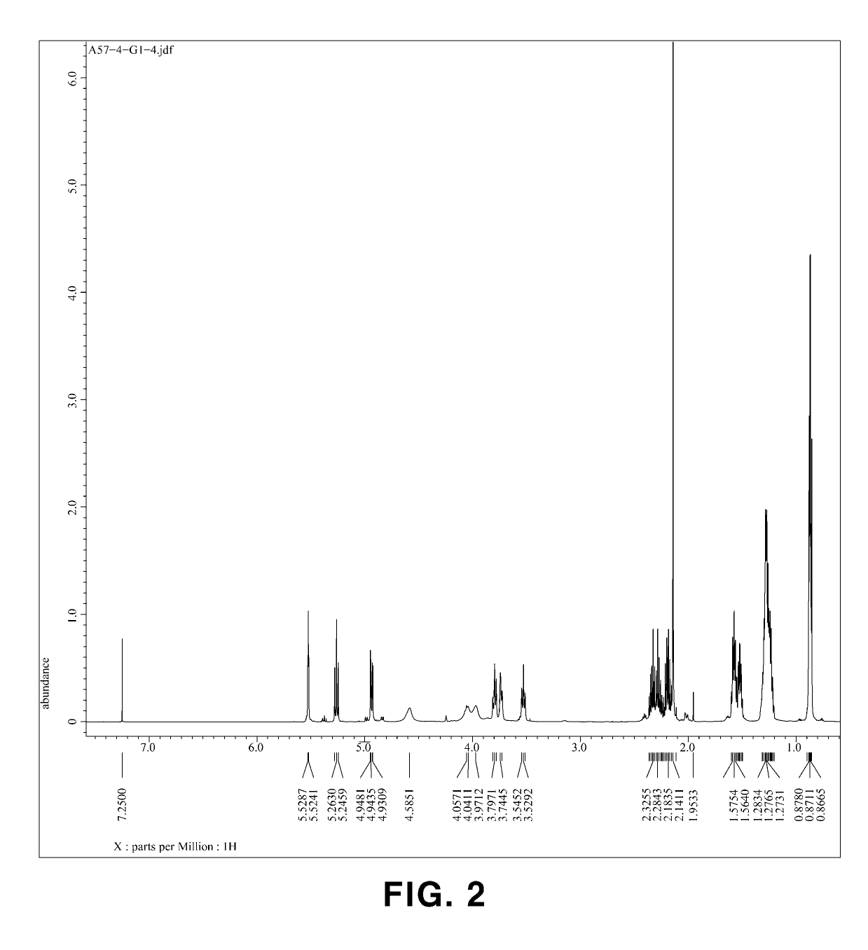

As a result of measuring a ¹H NMR spectrum by using CDCl₃ as a solvent (FIG. 2), six oxygenated methine protons at 5.52, 5.26, 4.94, 3.80, 3.73, and 3.53 ppm; eight methylene protons at 2.33/2.28, 2.19, 1.58, 1.52, and 1.2-1.4 ppm; and three methyl protons at 2.14, and 0.87(×2) ppm were observed. Also, three hydroxyl protons were observed at 4.59, 4.05, and 3.97 ppm.

Measurement and Interpretation of a ¹³C NMR Spectrum:

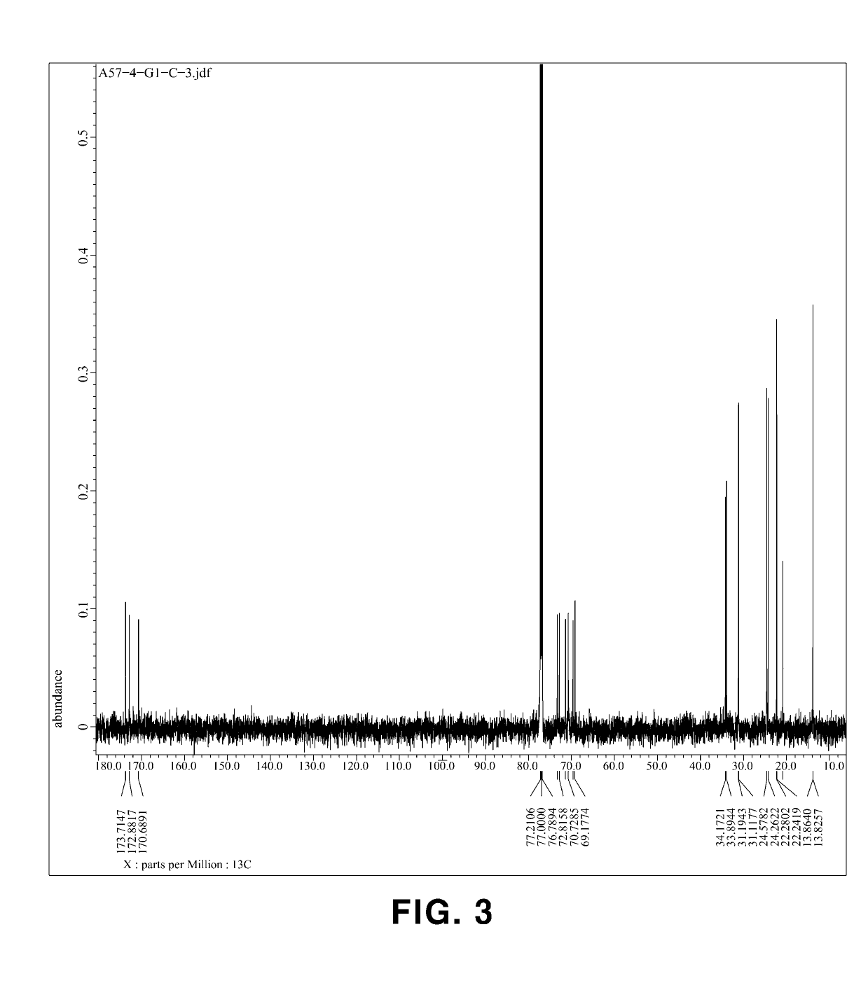

As a result of measuring a ¹³C NMR spectrum by using CDCl₃ as a solvent (FIG. 3), total twenty peaks were observed. That is, three ester carbonyl carbons at 173.7, 172.9, and 170.7 ppm; six oxygenated methine carbons at 73.3, 72.8, 71.4, 70.7, 69.7, and 69.2 ppm; eight methylene carbons at 34.2, 33.9, 31.2, 31.1, 24.6, 24.3, 22.3, and 22.2 ppm; and three methyl carbons at 20.8, 13.9, and 13.8 ppm were observed.

Measurement and Interpretation of a ¹H-¹H COSY Spectrum:

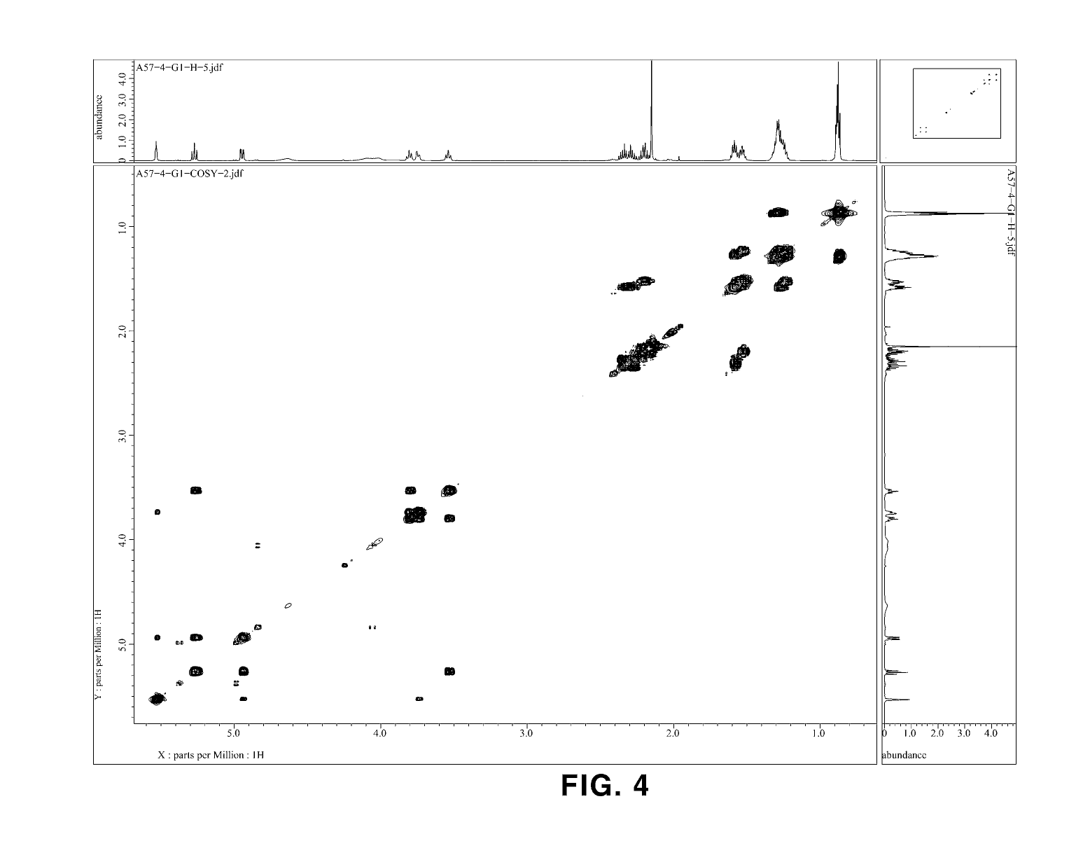

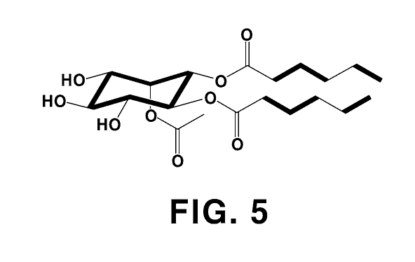

In order to investigate the chemical structure of compound A57-4-gly-1, a ¹H-¹H COSY spectrum which provides the information about correlations (³J_{H-H}) between coupled protons, was measured and interpreted (FIG. 4). As a result, the correlation between oxygenated methine protons was observed, suggesting the presence of an inositol moiety. It has been found that excepting the proton at 5.52 ppm from coupling constants of the protons, the rest of protons occupy an axial position. From these results, the inositol moiety constituting the compound of the present invention was identified as a myo-inositol. Further, four partial structures present in an acyl chain were identified (FIG. 5).

Measurement and Interpretation of a HMQC Spectrum:

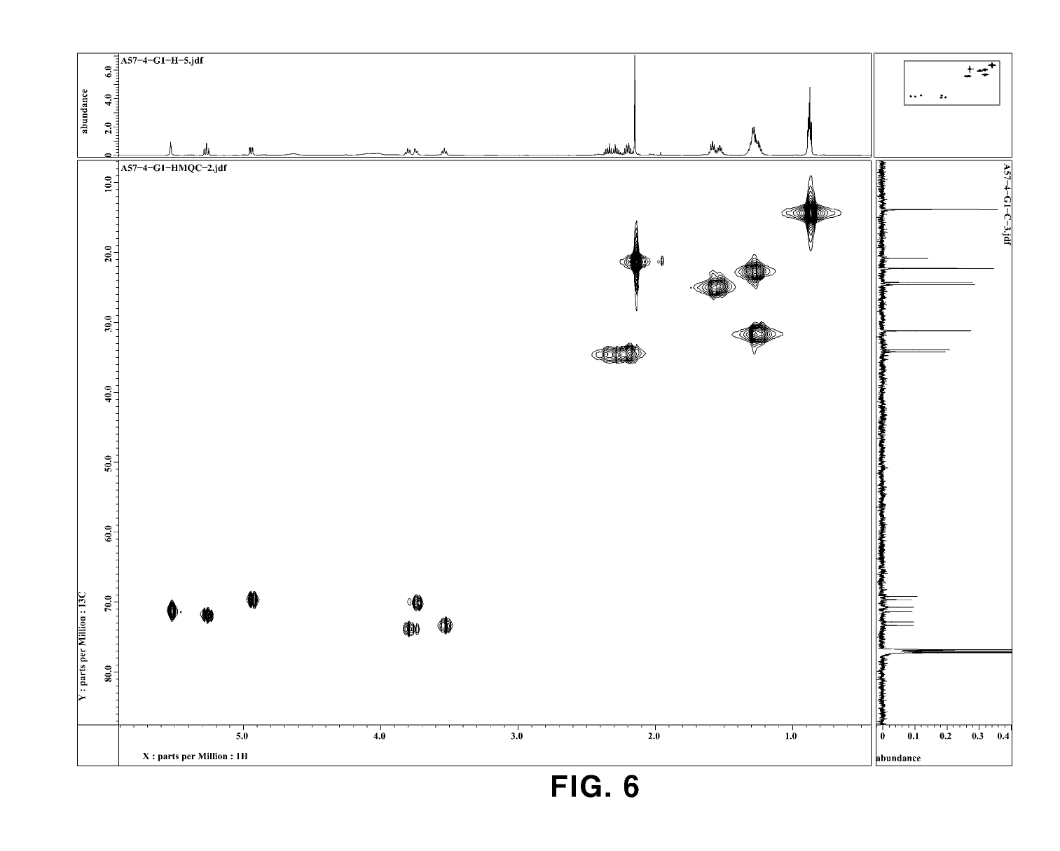

In order to investigate the chemical structure of compound A57-4-gly-1, a HMQC spectrum which provides the information about correlations (¹J_{C-H}) between hydrogen and carbon was measured and interpreted (FIG. 6). As a result, the correlation between all hydrogens and carbons constituting the compound of the present invention was identified.

Measurement and Interpretation of a HMBC Spectrum:

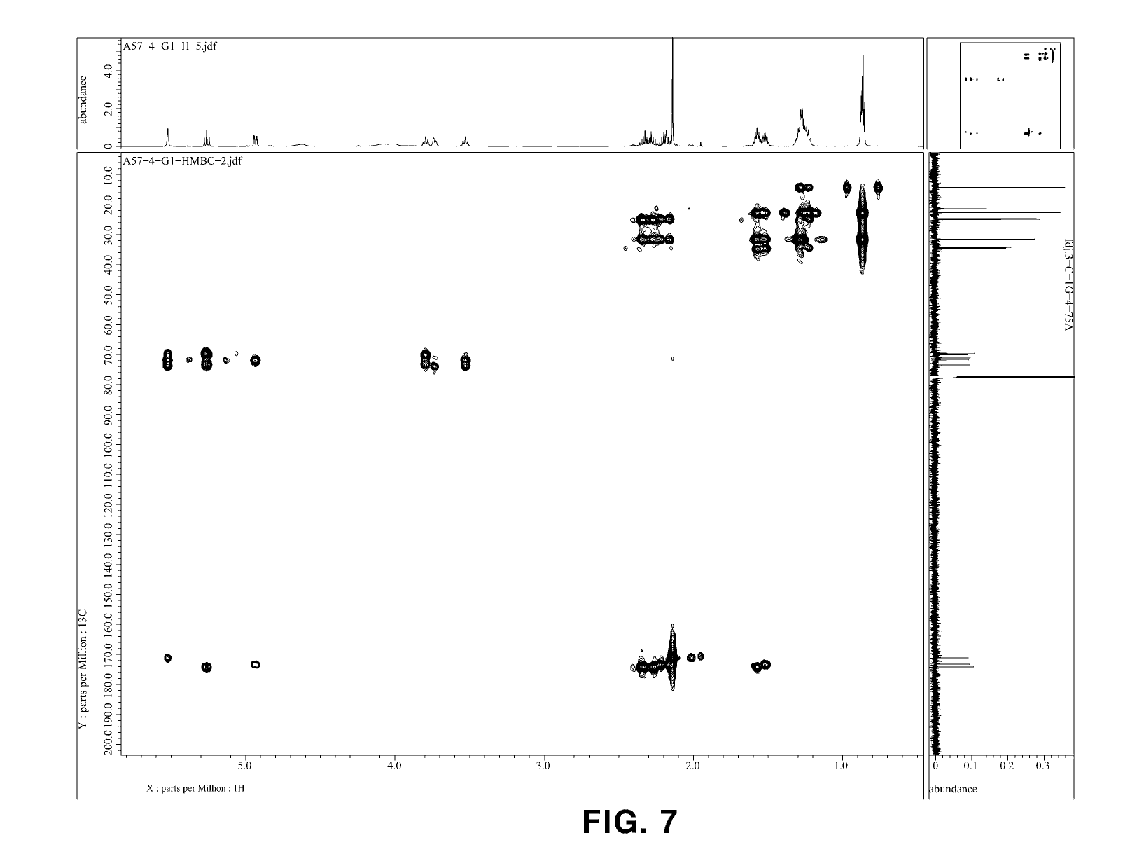

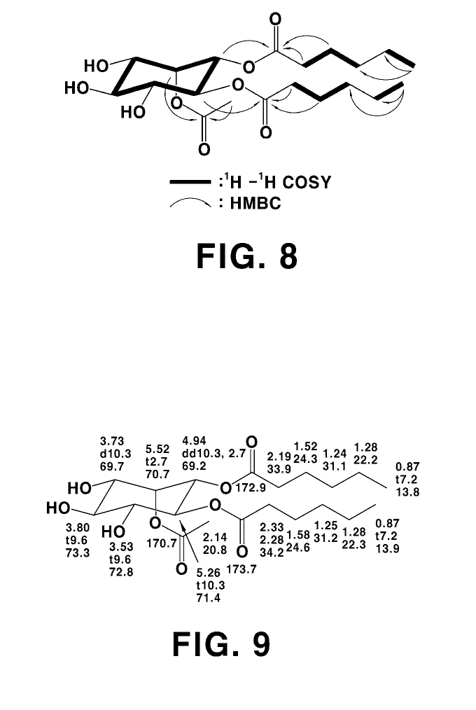

In order to investigate a chemical structure of compound A57-4-gly-1, a HMBC spectrum which provides the information about chemical shift (²J_{C-H}, ³J_{C-H}) of carbon atoms that are about 2-3 bonds away from the proton to which they correlate was measured and interpreted (FIG. 7). As a result, the long-range chemical shift correlation from methyl proton at 0.87 ppm to methylene carbon at 31.1 ppm, and that from methylene proton at 2.19, 1.52 ppm to ester carbonyl carbon at 172.9 ppm were observed. Also, the long-range chemical shift correlation from methyl proton at 0.87 ppm to methylene carbon at 31.2 ppm, and that from methylene proton at 2.33/2.28, and 1.58 ppm to ester carbonyl carbon at 173.7 ppm were observed. These results suggest the presence of two hexanoyl groups. In addition, the long-range chemical shift correlation from methyl proton at 2.14 ppm to ester carbonyl carbon at 170.7 ppm was observed, which identifies the presence of one acetyl group. Thus, it has been found that in the compound of the present invention, three acyl groups couple to the myo-inositol moiety. That is, there were the long-range chemical shift correlations from oxygenated methine protons at 5.52, 5.26, 4.94 ppm to ester carbonyl carbons at 170.7, 173.7, 172.9 ppm, respectively, which suggests that acetyl, hexanoyl, and hexanoyl groups couple to each position. The chemical structure of the compound according to the present invention was determined as illustrated in FIG. 8, and the reversal of each proton and carbon peak was shown in FIG. 9. As a result of searching databases and articles based on the chemical structure as identified above, it has been found that the compound of the present invention is novel.

Measurement and Interpretation of an ESI-Mass Spectrum:

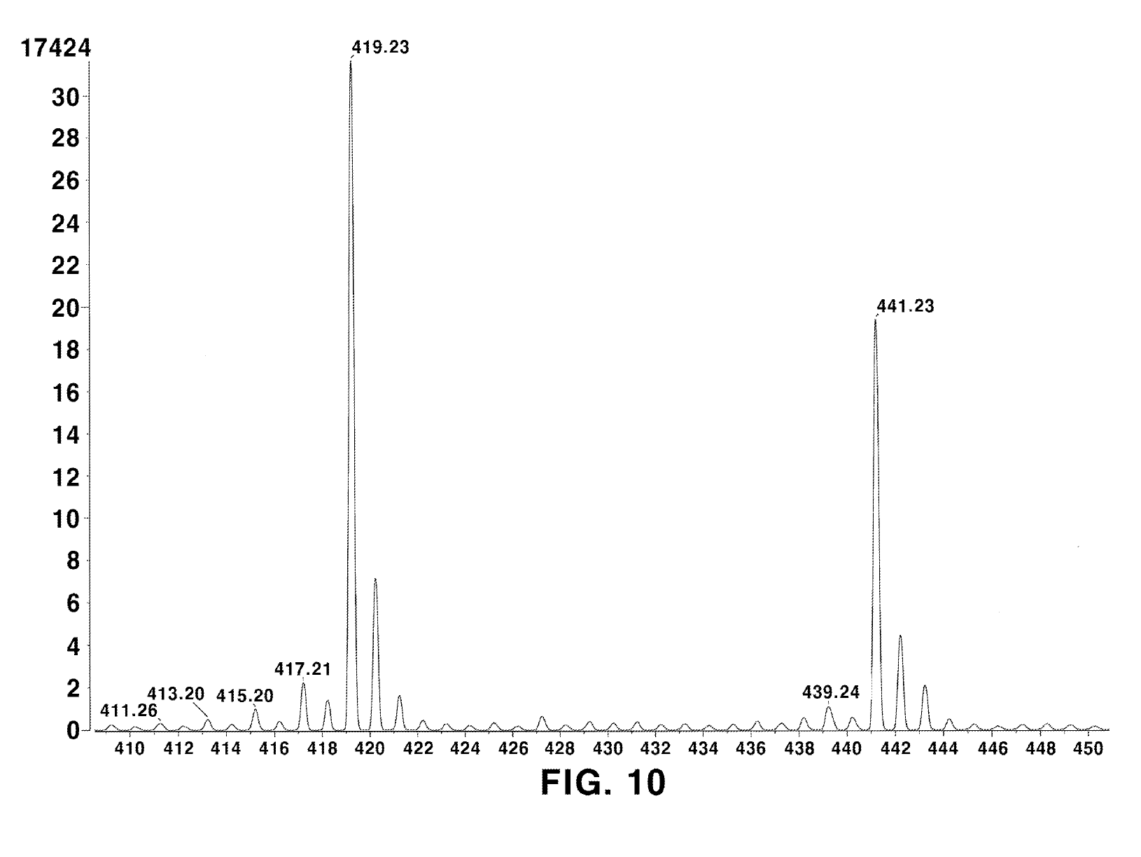

Finally, the chemical structure of the compound was confirmed by measuring its molecular weight and interpreting it with NMR. As shown in FIG. 10, [M+H]⁺ was observed at m/z 419, and [M+Na]⁺ was observed at m/z 441, which suggests that the compound of the present invention has a molecular weight of 418. In addition, a high-resolution ESI-mass spectrum was measured so as to confirm a molecular formula. As a result, [M+H]⁺ was observed at m/z 419.2252, which complied with a molecular formula of C₂₀H₃₅O₉(Δ−2.9 mmu). These results exactly coincided with the chemical structure interpreted by NMR.

(2) Compound A57-4-gly-2

In order to investigate a chemical structure of compound A57-4-gly-2, it was dissolved in CDCl₃, and subjected to ¹H NMR, ¹³C NMR, ¹H-¹H COSY, HMQC and HMBC analyses.

Measurement and Interpretation of a ¹H NMR Spectrum:

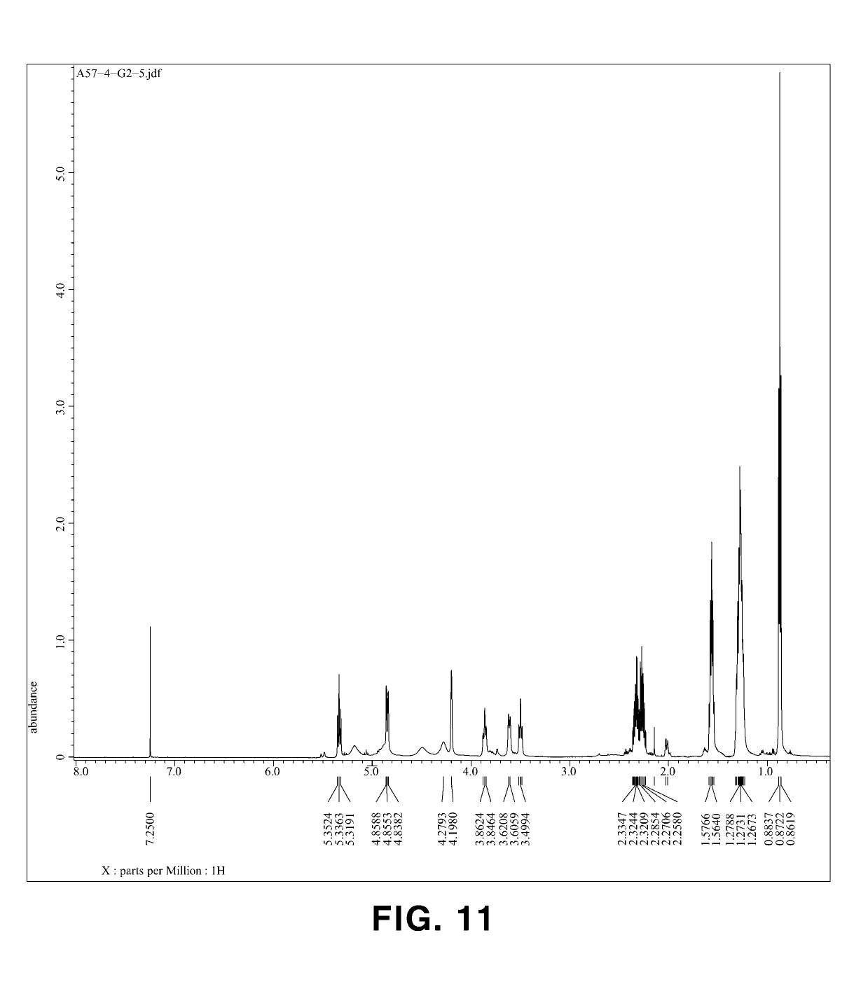

As a result of measuring a ¹H NMR spectrum by using CDCl₃ as a solvent (FIG. 11), six oxygenated methine protons at 5.34, 4.85, 4.20, 3.86, 3.61, and 3.50 ppm; eight methylene protons at 2.3212.27(×2), 1.56(×2), 1.28(×2), and 1.26(×2) ppm; and two methyl protons at 0.87(×2) ppm were observed. Also, four hydroxyl protons were observed at 5.17, 4.87, 4.49, and 4.28 ppm.

Measurement and Interpretation of a ¹³C NMR Spectrum:

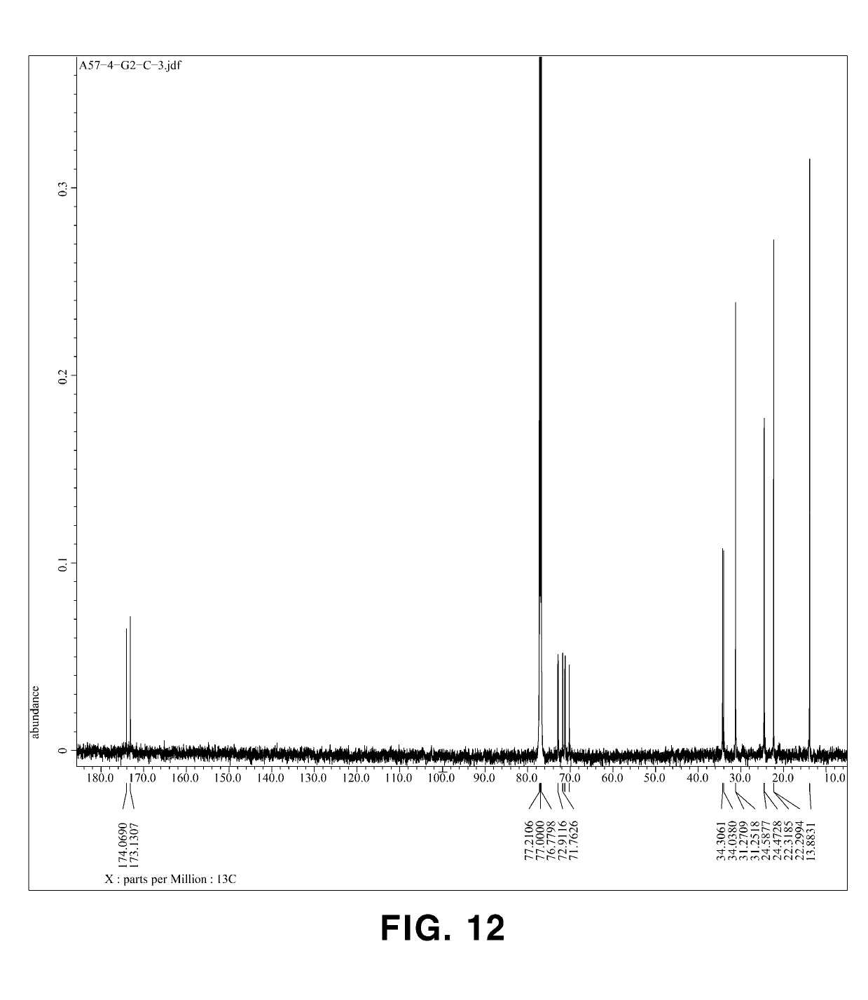

As a result of measuring a ¹³C NMR spectrum by using CDCl₃ as a solvent (FIG. 12), total eighteen peaks were observed. That is, two ester carbonyl carbons at 174.1, and 173.1 ppm; six oxygenated methine carbons at 72.9, 72.8, 71.8, 71.3, 71.2, and 70.2 ppm; eight methylene carbons at 34.3, 34.0, 31.3(×2), 24.6, 24.5, and 22.3(×2) ppm; and two methyl carbons at 13.9(×2) ppm were observed.

Measurement and Interpretation of a ¹H-¹H COSY Spectrum:

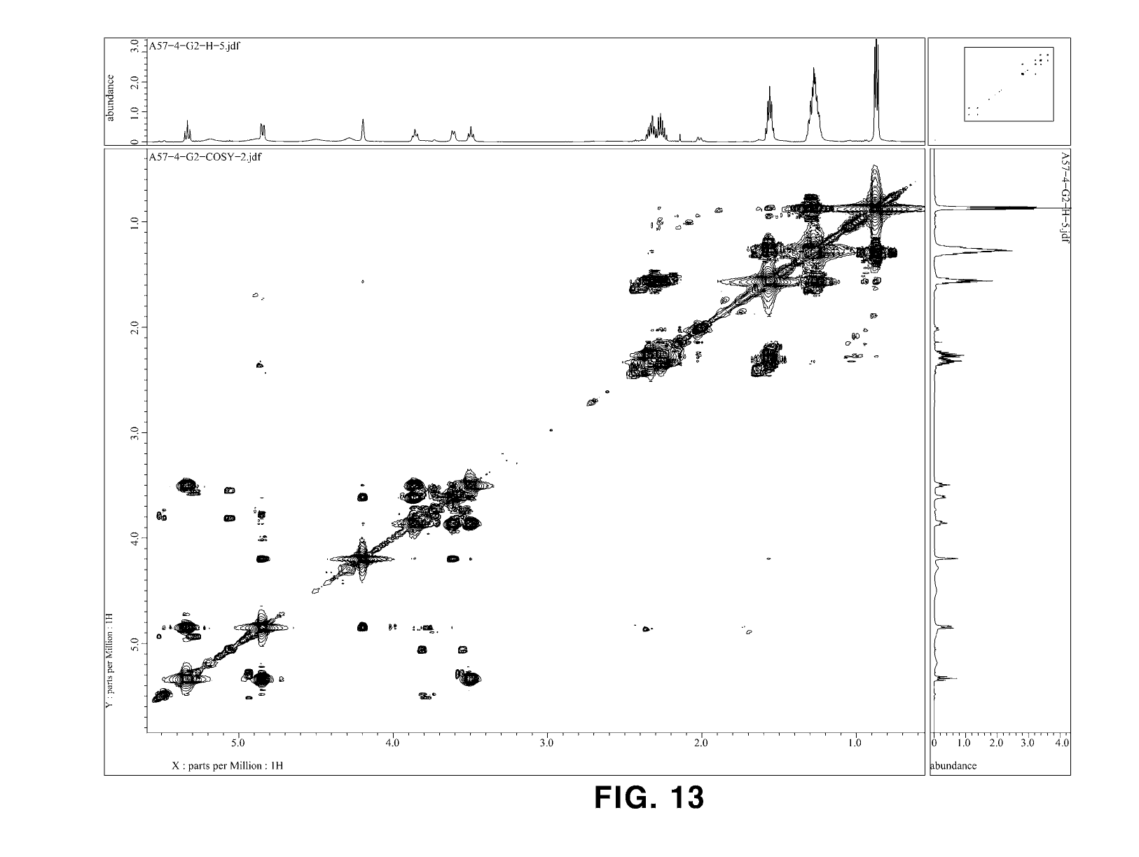

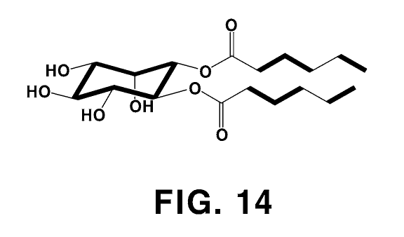

In order to investigate a chemical structure of compound A57-4-gly-2, a ¹H-¹H COSY spectrum which provides the information about correlations (³J_{H-H}) between coupled protons was measured and interpreted (FIG. 13). As a result, the correlation between oxygenated methine protons was observed, suggesting the presence of an inositol moiety. It has been found that excepting the proton at 4.20 ppm from coupling constants of the protons, the rest of protons occupy an axial position. From these results, the inositol moiety constituting the compound of the present invention was identified as a myo-inositol. Further, four partial structures present in an acyl chain were identified (FIG. 14).

### Measurement and Interpretation of a HMQC Spectrum:

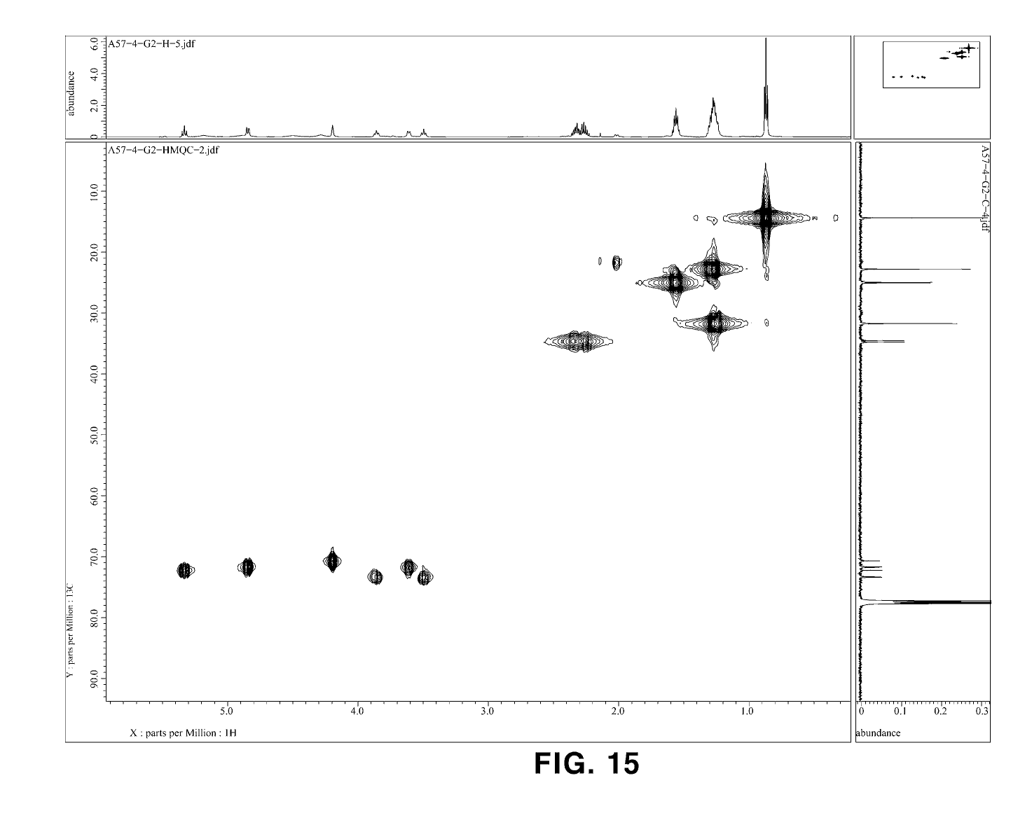

In order to investigate a chemical structure of compound A57-4-gly-2, a HMQC spectrum which provides the information about correlations (¹J_{C-H}) between hydrogen and carbon was measured and interpreted (FIG. 15). As a result, the correlation between all hydrogens and carbons constituting the compound of the present invention was identified.

Measurement and Interpretation of a HMBC Spectrum:

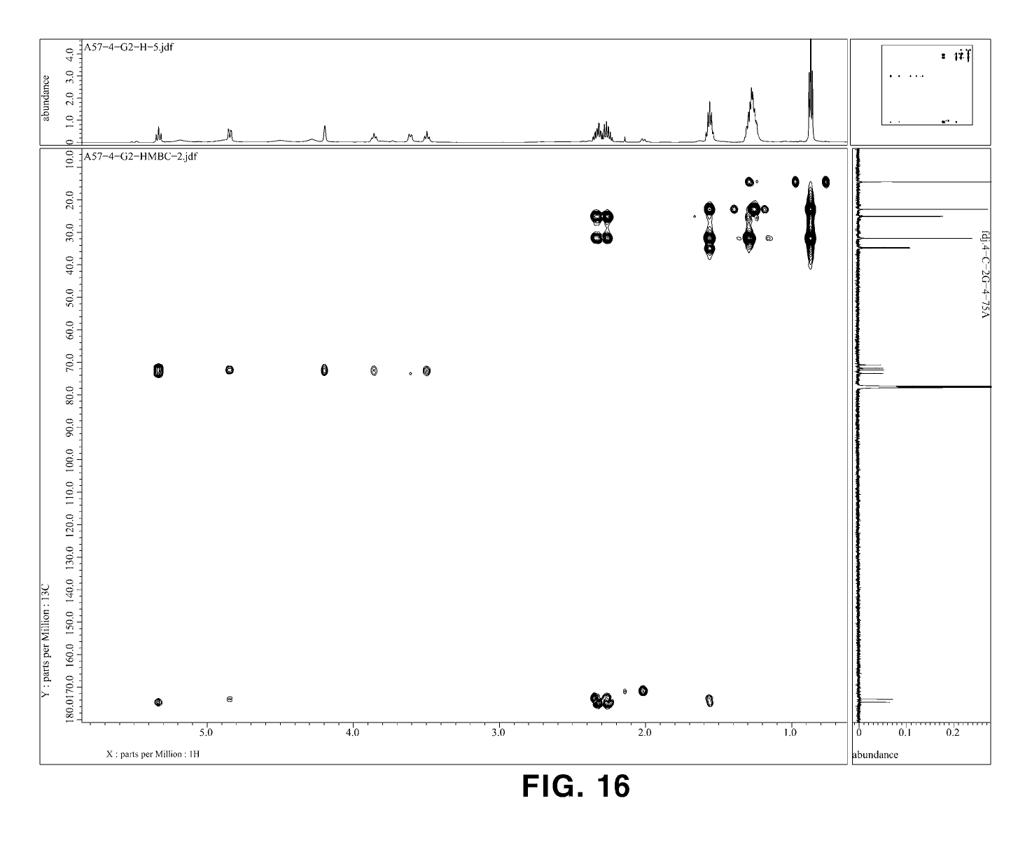

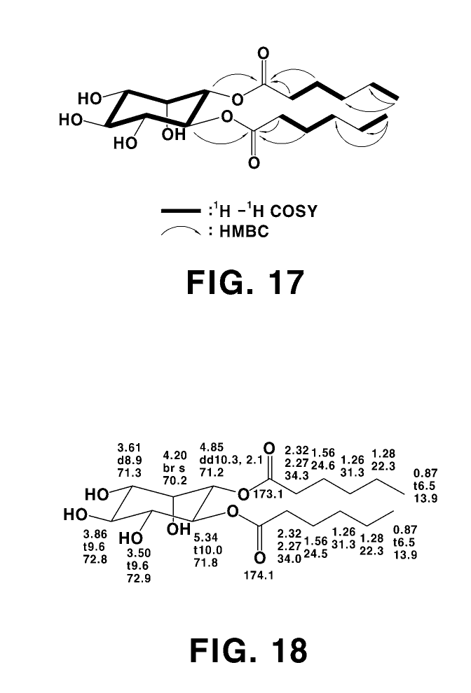

In order to investigate a chemical structure of compound A57-4-gly-2, a HMBC spectrum which provides the information about chemical shift (²J_{C-H}, ³J_{C-H}) of carbon atoms that are about 2-3 bonds away from the proton to which they correlate was measured and interpreted (FIG. 16). As a result, the long-range chemical shift correlation from methyl proton at 0.87 ppm to methylene carbon at 31.1 ppm, and that from methylene proton at 2.32/2.27, and 1.56 ppm to ester carbonyl carbon at 174.1 and 173.1 ppm were observed, which suggests the presence of two hexanoyl groups. Also, the long-range chemical shift correlation from oxygenated methine protons at 5.34, and 4.85 ppm to ester carbonyl carbons at 174.1, and 173.1 ppm was observed, which suggests that a hexanoyl group coupled to each position. Thus, it has been found that in the compound of the present invention, two hexanoyl groups couple to the myo-inositol moiety. The chemical structure of the compound according to the present invention was determined as illustrated in FIG. 17, and the reversal of each proton and carbon peak was shown in FIG. 18. As a result of searching databases and articles based on the chemical structure as identified above, it has been found that the compound of the present invention is novel.

Measurement and Interpretation of an ESI-Mass Spectrum:

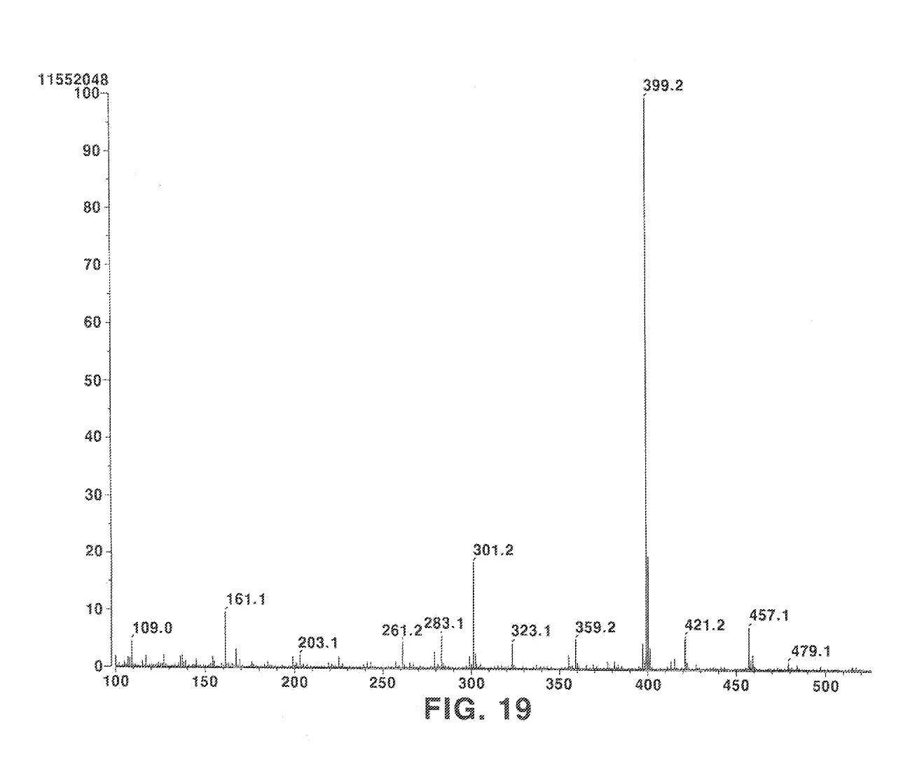

Finally, the chemical structure of the compound was confirmed by measuring its molecular weight and interpreting it with NMR. As shown in FIG. 19, [M+Na]⁺ was observed at m/z 399, which suggests that the compound of the present invention has a molecular weight of 376. In addition, a high-resolution ESI-mass spectrum was measured so as to confirm a molecular formula. As a result, [M+Na]⁺ was observed at m/z 399.2012, which complied with a molecular formula of C₁₈H₃₂O₈Na(Δ+1.7 mmu). These results exactly coincided with the chemical structure interpreted by NMR.

(3) Compound A57-4-gly-3

In order to investigate a chemical structure of compound A57-4-gly-3, it was dissolved in CDCl₃, and subjected to ¹H NMR, ¹³C NMR, ¹H-¹H COSY, HMQC and HMBC analyses.

Measurement and Interpretation of a ¹H NMR Spectrum:

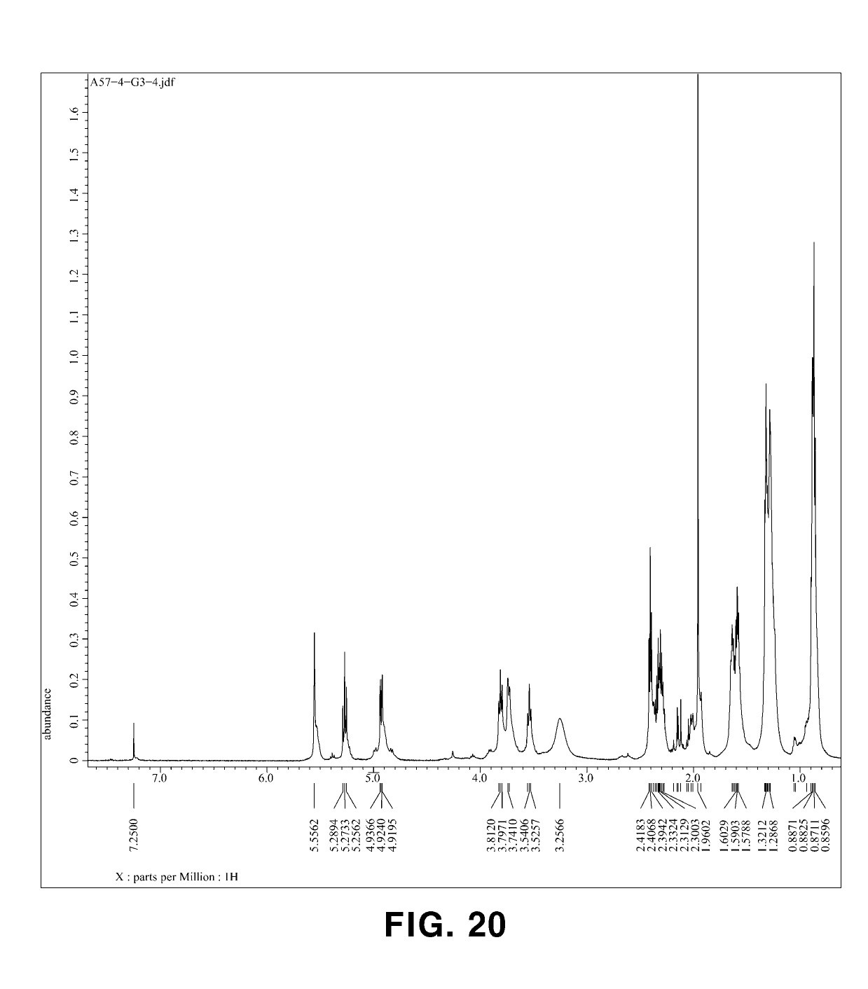

As a result of measuring a ¹H NMR spectrum by using CDCl₃ as a solvent (FIG. 20), six oxygenated methine protons at 5.56, 5.28, 4.94, 3.81, 3.73, and 3.54 ppm; eight methylene protons at 2.41, 2.32, 1.64, 1.59, 1.32(×2), and 1.29(×2) ppm; and three methyl protons at 1.96, 0.88, and 0.87 ppm were observed. Also, three hydroxyl protons were observed at 3.58, 3.17, and 3.13 ppm.

Measurement and Interpretation of a ¹³C NMR Spectrum:

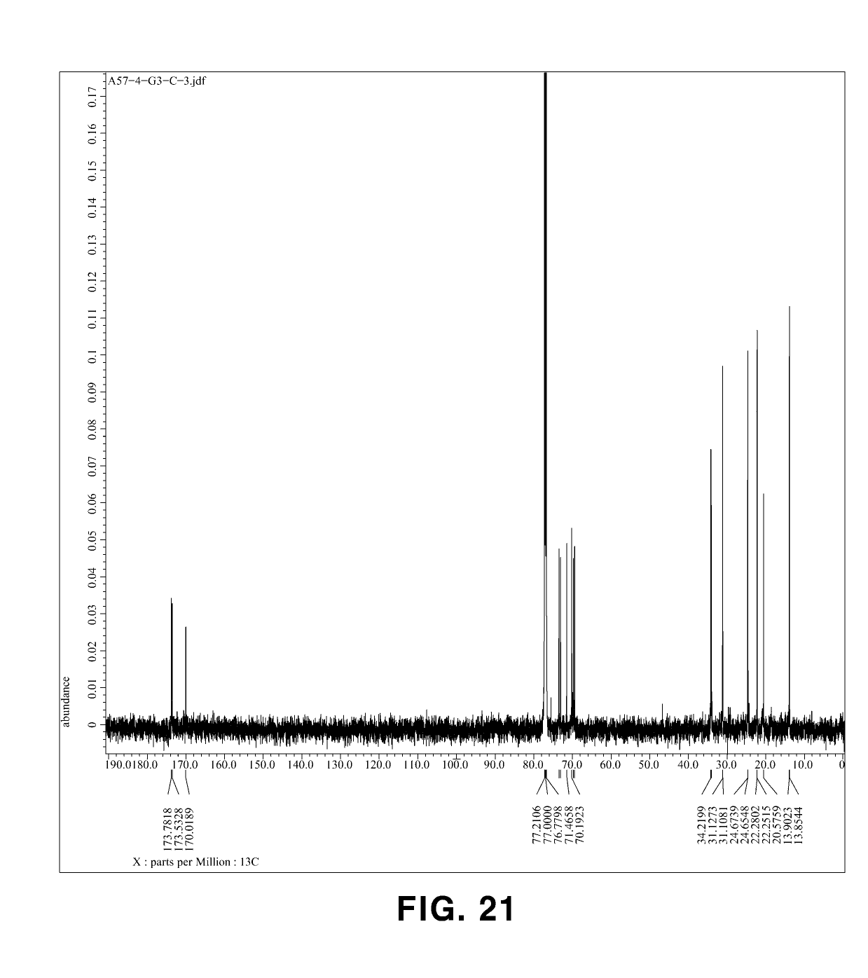

As a result of measuring a ¹³C NMR spectrum by using CDCl₃ as a solvent (FIG. 21), total twenty peaks were observed. That is, three ester carbonyl carbons at 173.8, 173.5, and 170.0 ppm; six oxygenated methine carbons at 73.5, 73.1, 71.5, 70.2, 69.8, and 69.5 ppm; eight methylene carbons at 34.2, 33.9, 31.2, 31.1, 24.6, 24.3, 22.3, and 22.2 ppm; and three methyl carbons at 20.6, and 13.9(×2) ppm were observed.

Measurement and Interpretation of a ¹H-¹H COSY Spectrum:

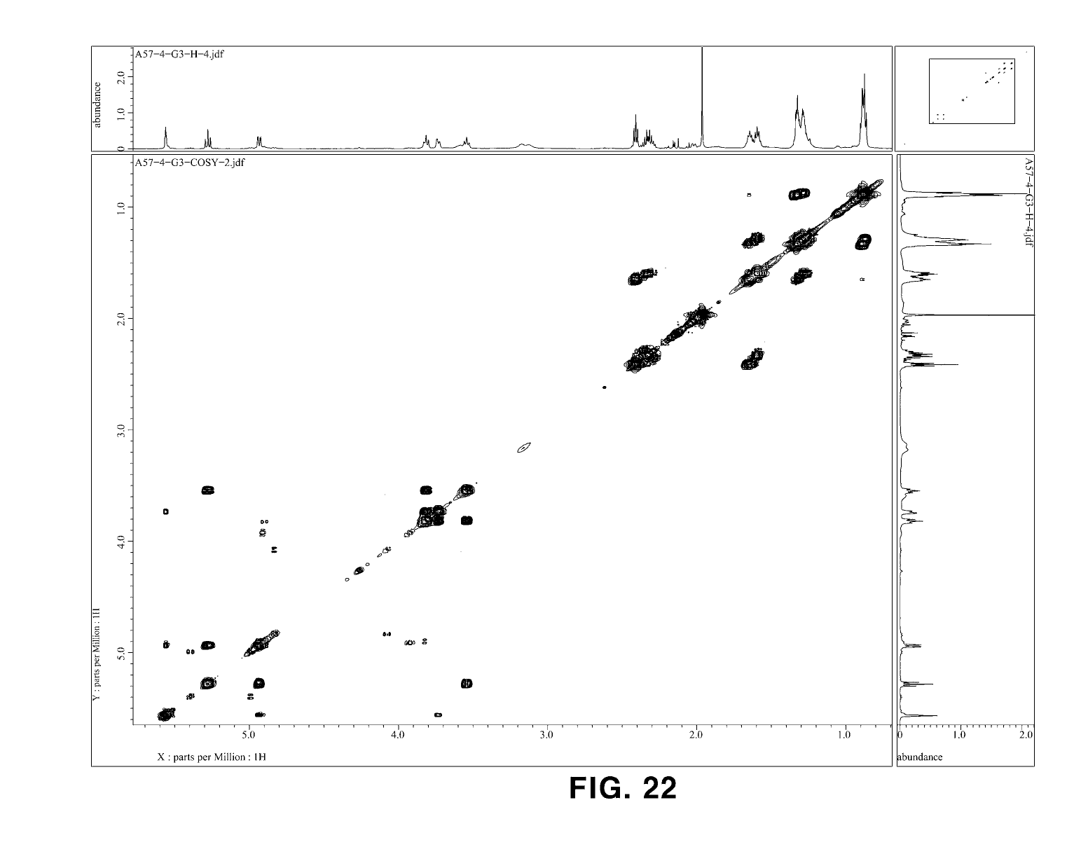

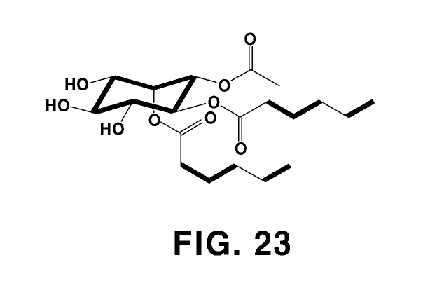

In order to investigate a chemical structure of compound A57-4-gly-3, a ¹H-¹H COSY spectrum which provides the information about correlations (³J_{H-H}) between coupled protons was measured and interpreted (FIG. 22). As a result, the correlation between oxygenated methine protons was observed, suggesting the presence of an inositol moiety. It has been found that excepting the proton at 5.56 ppm from coupling constants of the protons, the rest of protons occupy an axial position. From these results, the inositol moiety constituting the compound of the present invention was identified as a myo-inositol. Further, four partial structures present in an acyl chain were identified (FIG. 23).

Measurement and Interpretation of a HMQC Spectrum:

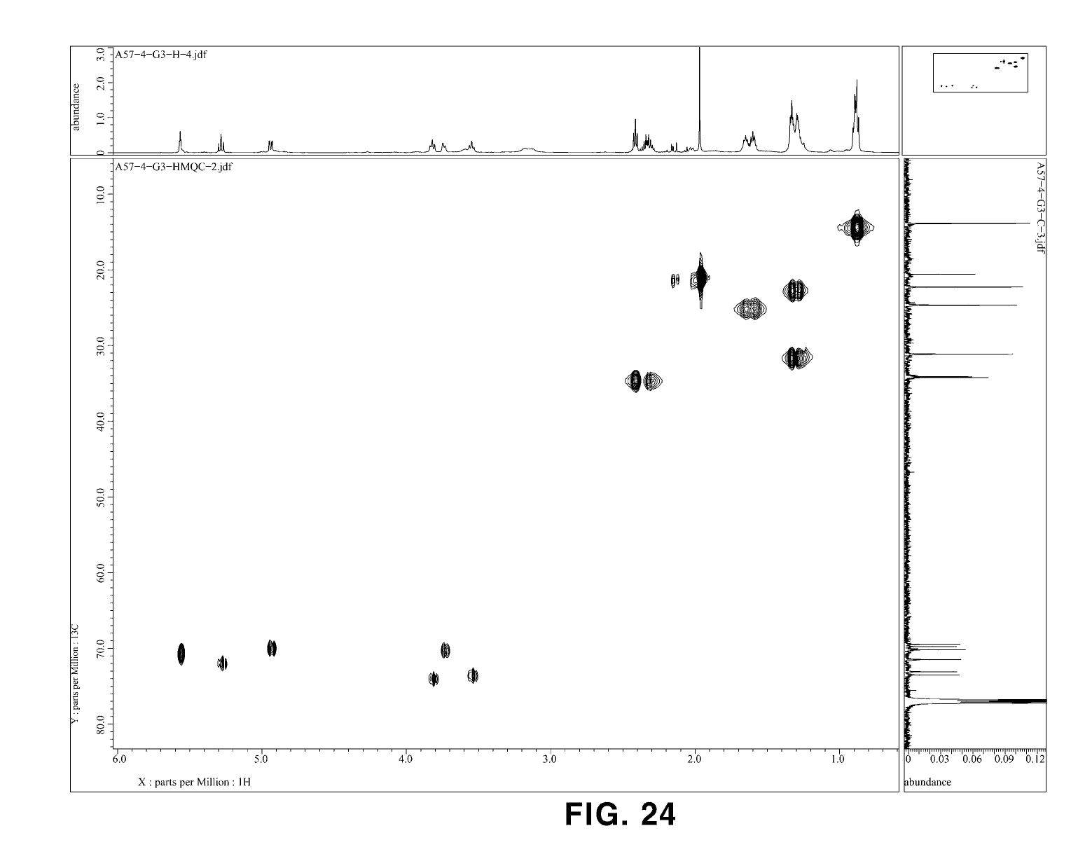

In order to investigate a chemical structure of compound A57-4-gly-3, a HMQC spectrum which provides the information about correlations (¹J_{C-H}) between hydrogen and carbon was measured and interpreted (FIG. 24). As a result, the correlation between all hydrogens and carbons constituting the compound of the present invention was identified.

Measurement and Interpretation of a HMBC Spectrum:

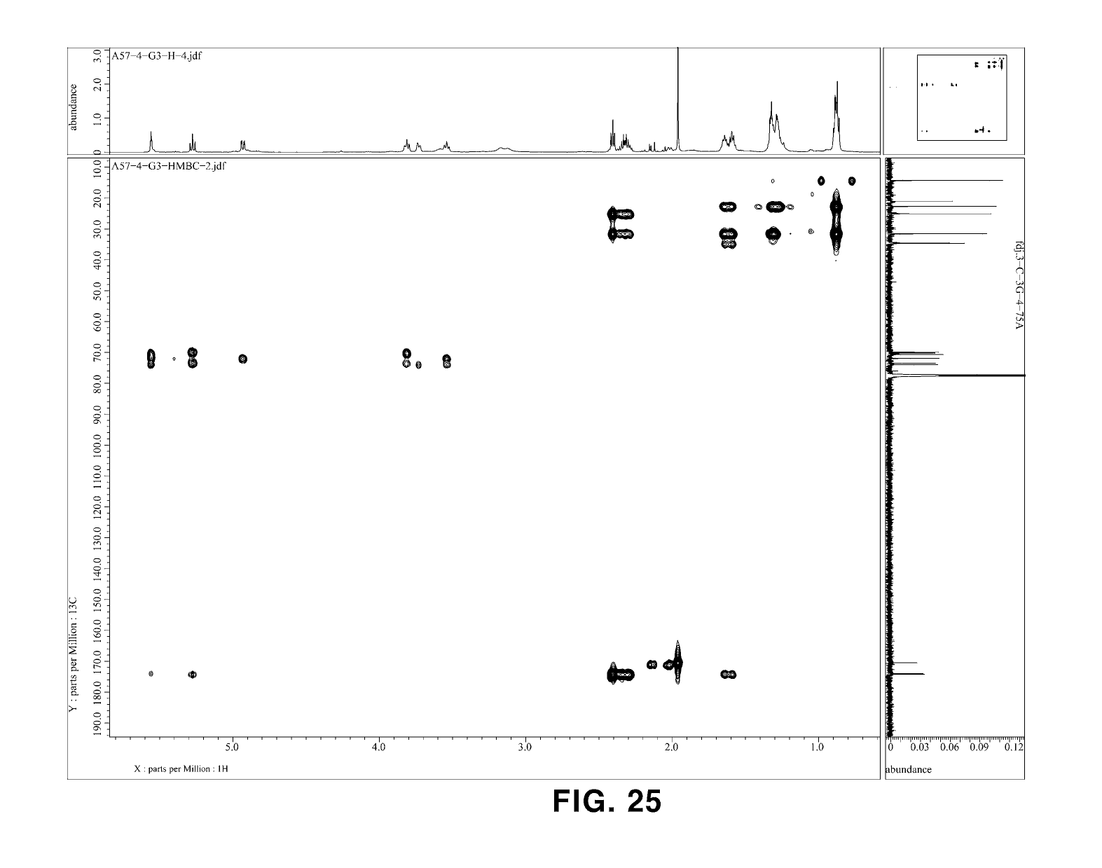

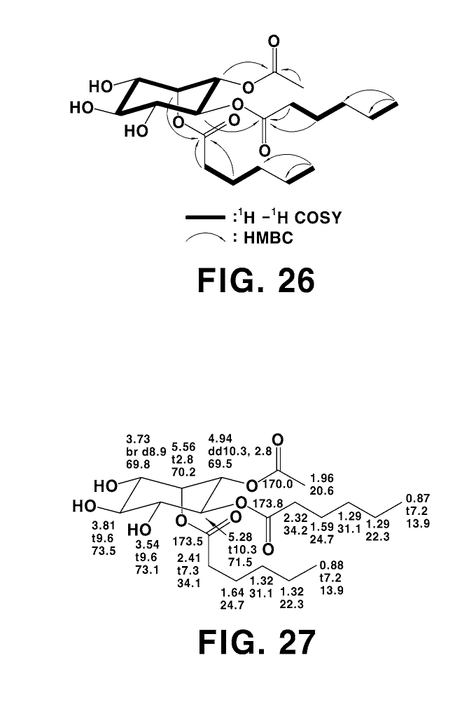

In order to investigate a chemical structure of compound A57-4-gly-3, a HMBC spectrum which provides the information about chemical shift (²J_{C-H}, ³J_{C-H}) of carbon atoms that are about 2-3 bonds away from the proton to which they correlate was measured and interpreted (FIG. 25). As a result, the long-range chemical shift correlation from methyl protons at 0.87 and 0.88 ppm to methylene carbon at 31.1 ppm, and that from methylene protons at 2.41 and 2.32 ppm to ester carbonyl carbon at 173.5 and 173.8 ppm were observed, which suggests the presence of two hexanoyl groups. Also, the long-range chemical shift correlation from methyl protons at 1.96 ppm to ester carbonyl carbon at 170.0 ppm was observed, which confirms the presence of one acetyl group. Thus, it has been found that in the compound of the present invention, three acyl groups couple to the myo-inositol moiety. For the substitution position of each acyl group, the long-range chemical shift correlation from oxygenated methine protons at 5.56, 5.28, and 4.94 ppm to ester carbonyl carbons at 173.5, 173.8, and 170.0 ppm, respectively, was observed, which suggests that acetyl, hexanoyl, and hexanoyl groups couple to each position. Therefore, the chemical structure of the compound according to the present invention was determined as illustrated in FIG. 26, and the reversal of each proton and carbon peak was shown in FIG. 27. As a result of searching databases and articles based on the chemical structure as identified above, it has been found that the compound of the present invention is novel.

Measurement and Interpretation of an ESI-Mass Spectrum:

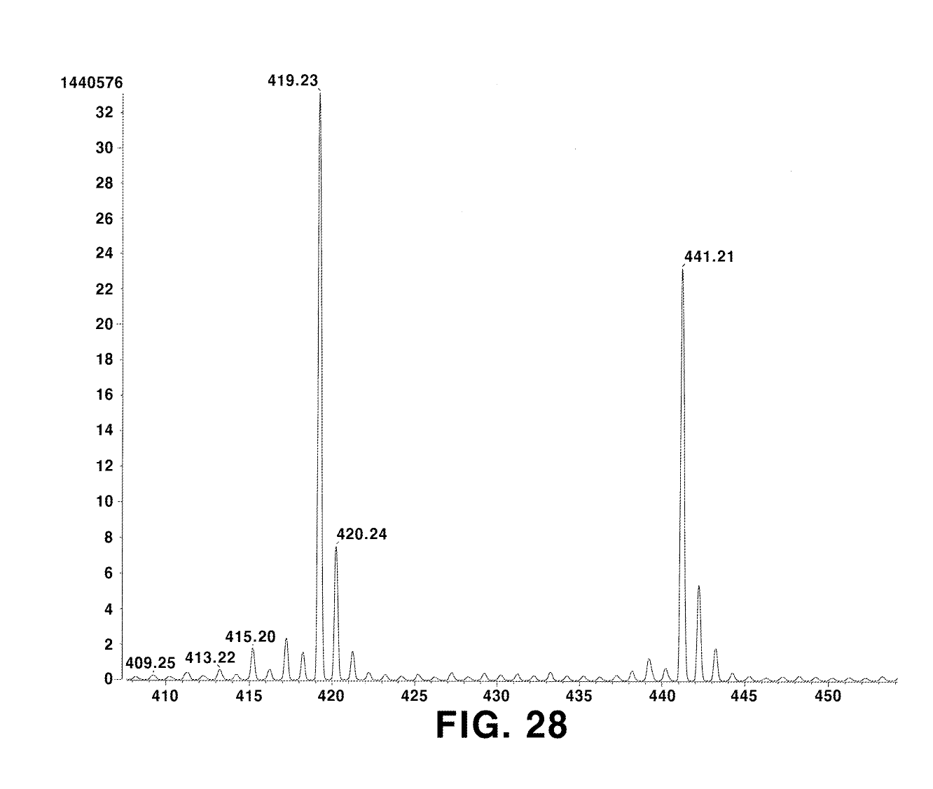

Finally, the chemical structure of the compound was confirmed by measuring its molecular weight and interpreting it with NMR. As shown in FIG. 28, [M+H]⁺ was observed at m/z 419, and [M+Na]⁺ was observed at m/z 441, which suggests that the compound of the present invention has a molecular weight of 418. In addition, a high-resolution ESI-mass spectrum was measured so as to confirm a molecular formula. As a result, [M+H]⁺ was observed at m/z 419.2256, which complied with a molecular formula of C₂₀H₃₅O₉(Δ−2.5 mmu). These results exactly coincided with the chemical structure interpreted by NMR.

(4) Compound A57-4-gly-4

In order to investigate a chemical structure of compound A57-4-gly-4, It was dissolved in CDCl₃, and subjected to ¹H NMR, ¹³C NMR, ¹H-¹H COSY, HMQC and HMBC analyses.

Measurement and Interpretation of a ¹H NMR Spectrum:

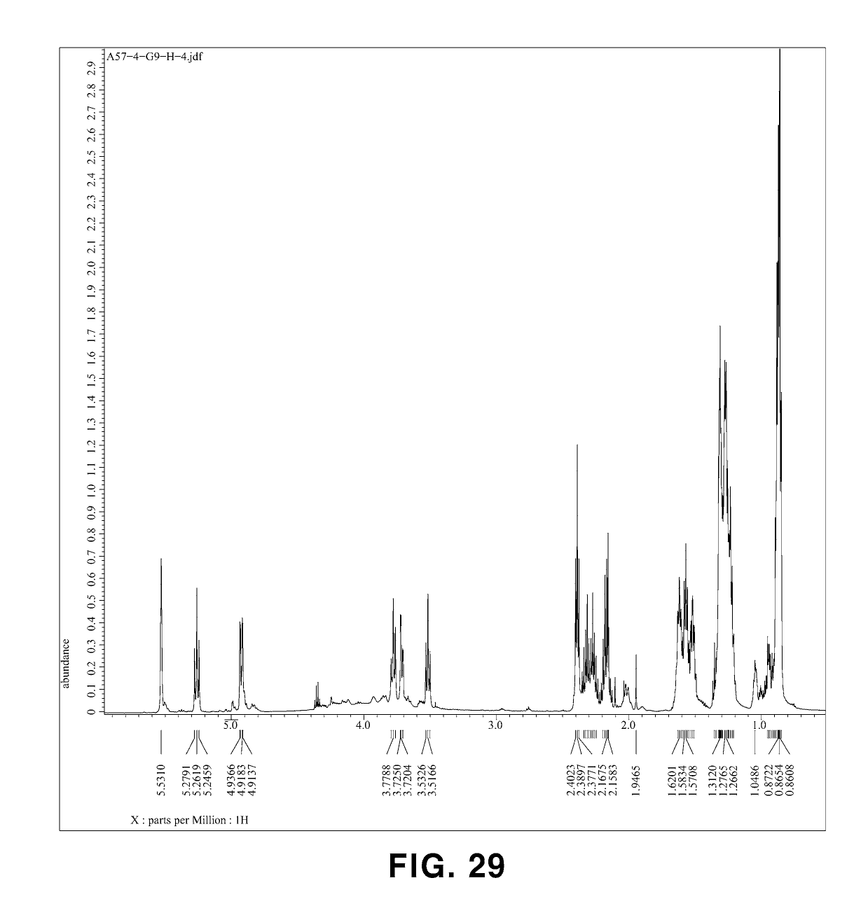

As a result of measuring a ¹H NMR spectrum by using CDCl₃ as a solvent (FIG. 29), six oxygenated methine protons at 5.53, 5.26, 4.93, 3.78, 3.71, and 3.52 ppm; twelve methylene protons at 2.39, 2.33/2.26, 2.17, 1.62, 1.57, 1.52, 1.31, 1.27, 1.25, and 1.2-1.4(×3) ppm; and three methyl protons at 0.8-0.9(×3) ppm were observed.

Measurement and Interpretation of a ¹³C NMR Spectrum:

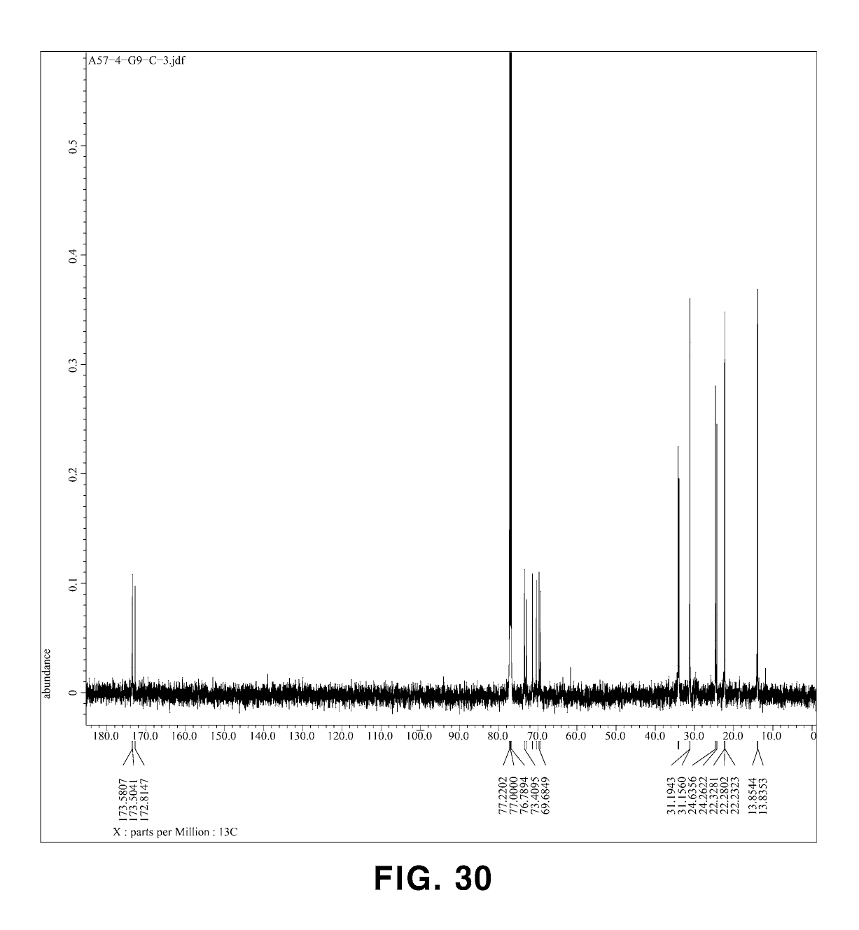

As a result of measuring a ¹³C NMR spectrum by using CDCl₃ as a solvent (FIG. 30), total twenty-four peaks were observed. That is, three ester carbonyl carbons at 173.6, 173.5, and 172.8 ppm; six oxygenated methine carbons at 73.4, 72.9, 71.4, 70.4, 69.7, and 69.3 ppm; twelve methylene carbons at 34.2, 34.0, 33.9, 31.2(×2), 31.1, 24.6(×2), 24.3, 22.3(×2), and 22.2 ppm; and three methyl carbons at 13.9(×2), and 13.8 ppm were observed.

Measurement and Interpretation of a ¹H-¹H COSY Spectrum:

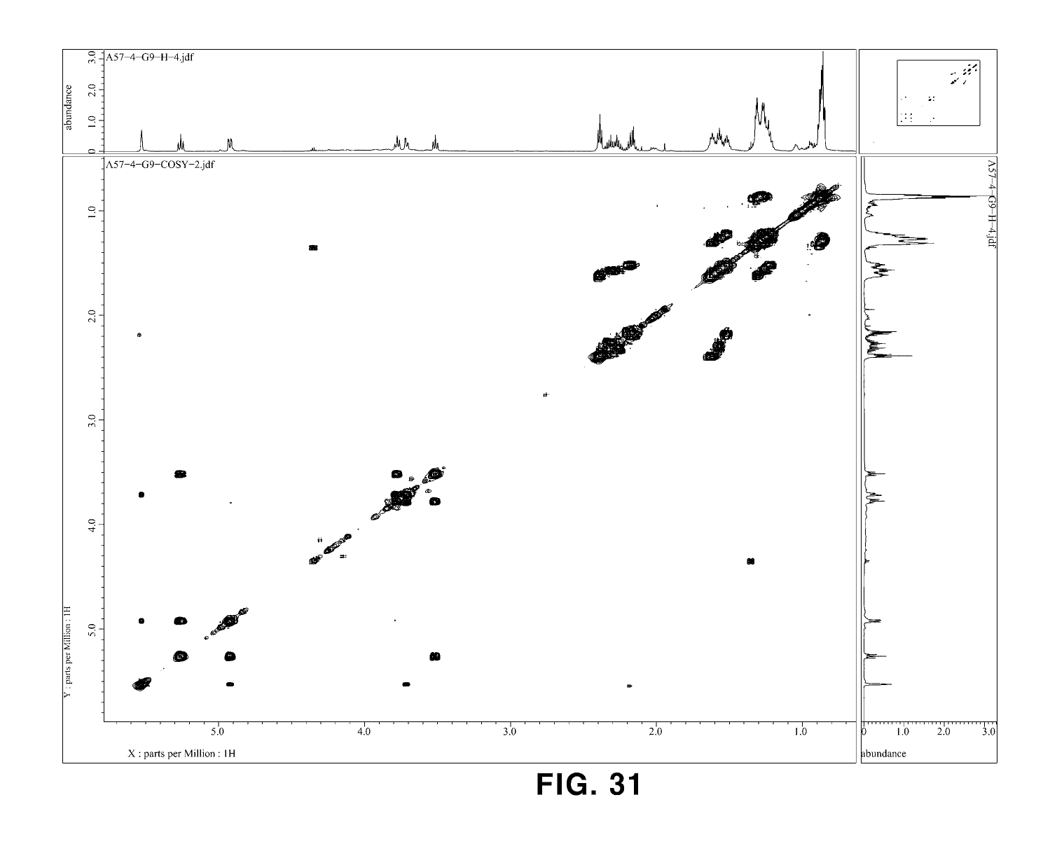

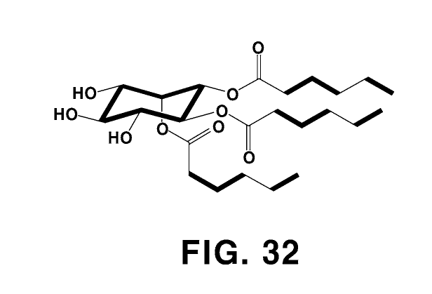

In order to investigate a chemical structure of compound A57-4-gly-4, a ¹H-¹H COSY spectrum which provides the information about correlations (³J_{H-H}) between coupled protons was measured and interpreted (FIG. 31). As a result, the correlation between oxygenated methine protons was observed, suggesting the presence of an inositol moiety. It has been found that excepting the proton at 5.53 ppm from coupling constants of the protons, the rest of protons occupy an axial position. From these results, the inositol moiety constituting the compound of the present invention was identified as a myo-inositol. Further, six partial structures present in an acyl chain were identified (FIG. 32).

Measurement and Interpretation of a HMQC Spectrum:

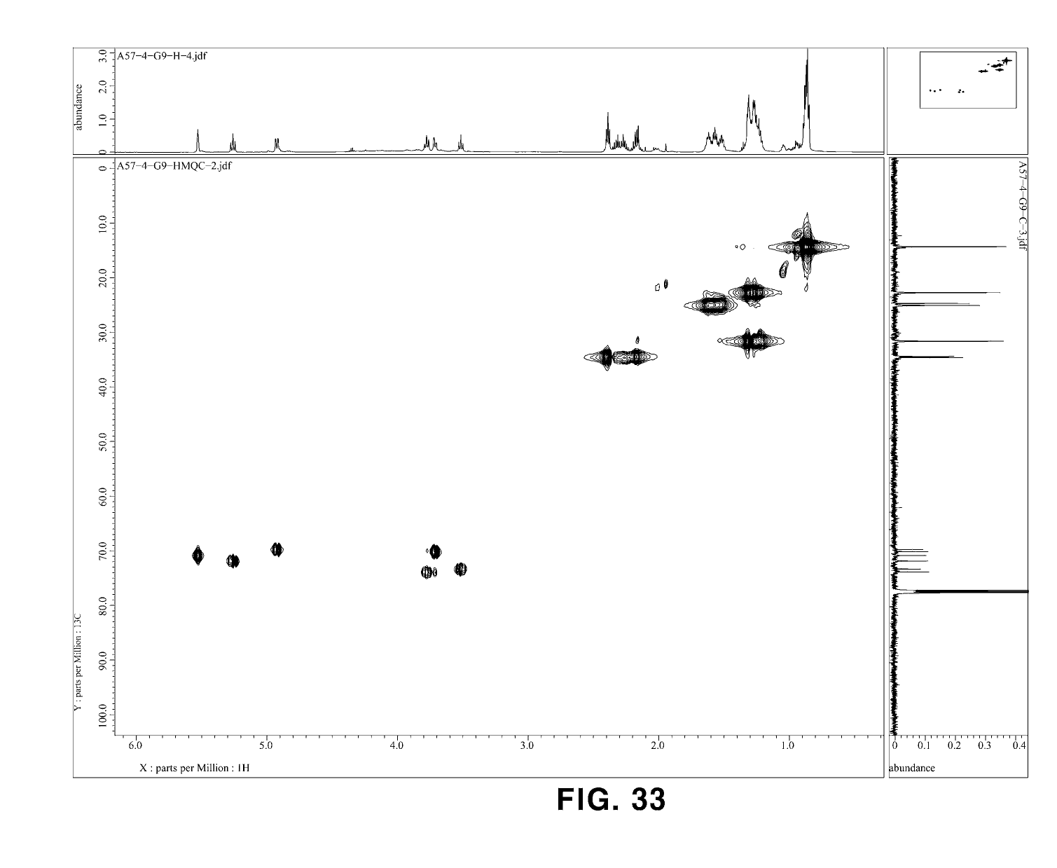

In order to investigate a chemical structure of compound A57-4-gly-4, a HMQC spectrum which provides the information about correlations (¹J_{C-H}) between hydrogen and carbon was measured and interpreted (FIG. 33). As a result, the correlation between all hydrogens and carbons constituting the compound of the present invention was identified.

Measurement and Interpretation of a HMBC Spectrum:

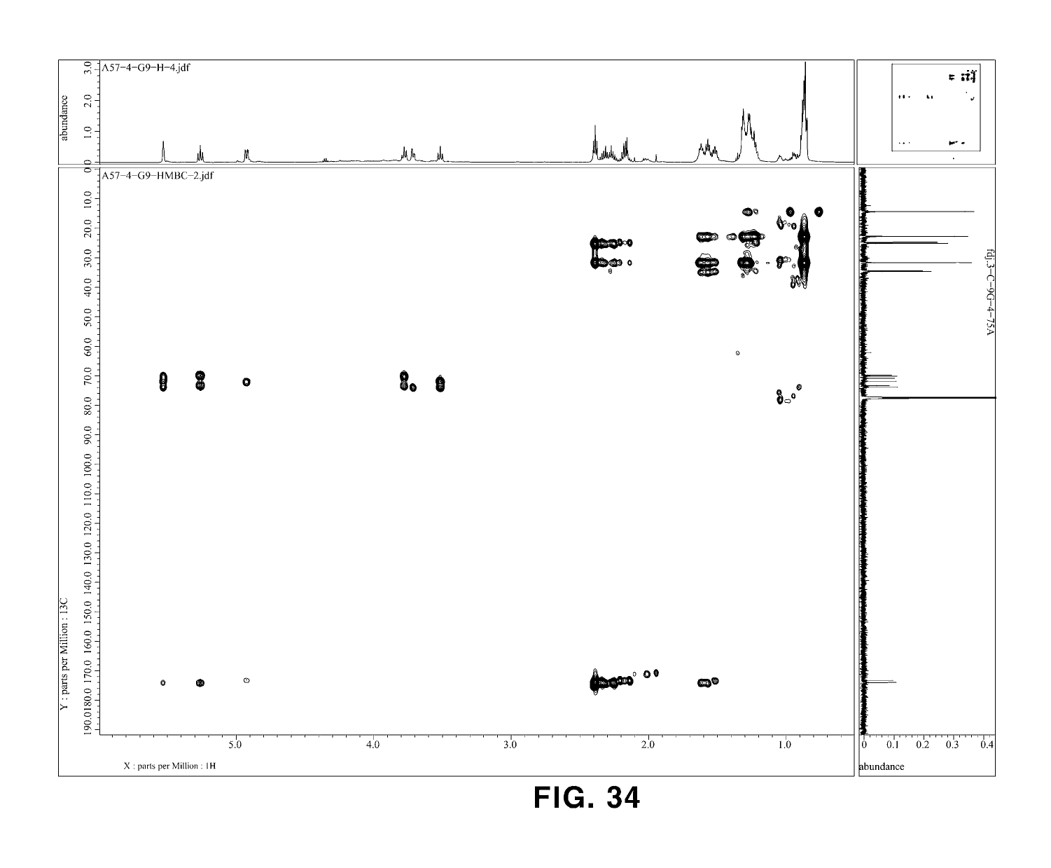

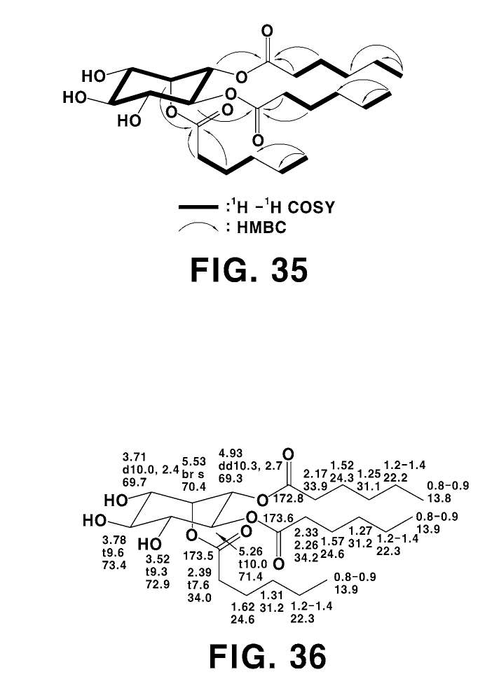

In order to investigate a chemical structure of compound A57-4-gly-4, a HMBC spectrum which provides the information about chemical shift (²J_{C-H}, ³J_{C-H}) of carbon atoms that are about 2-3 bonds away from the proton to which they correlate was measured and interpreted (FIG. 34). As a result, the long-range chemical shift correlation from three methyl protons at 0.8-0.9 ppm to three methylene carbons at 31.1 and 31.2 ppm, that from methylene protons at 2.39, and 1.62 ppm to ester carbonyl carbon at 173.5 ppm, that from methylene protons at 2.33/2.26, and 1.57 ppm to ester carbonyl carbon at 173.6 ppm, and that from methylene protons at 2.17, and 1.52 ppm to ester carbonyl carbon at 172.8 ppm were observed. These results suggest that three hexanoyl groups are present in the compound of the present invention. In addition, the long-range chemical shift correlation from oxygenated methine protons at 5.53, 5.26, and 4.93 ppm to ester carbonyl carbons at 173.5, 173.6, and 172.8 ppm, respectively, was observed, which confirms the position to which each hexanoyl group coupled. Therefore, it has been found that the compound of the present invention has a chemical structure where three hexanoyl groups couple to the myo-inositol moiety successively. The chemical structure of the compound according to the present invention was determined as illustrated in FIG. 35, and the reversal of each proton and carbon peak was shown in FIG. 36. As a result of searching databases and articles based on the chemical structure as identified above, it has been found that the compound of the present invention is novel.

Measurement and Interpretation of an ESI-Mass Spectrum:

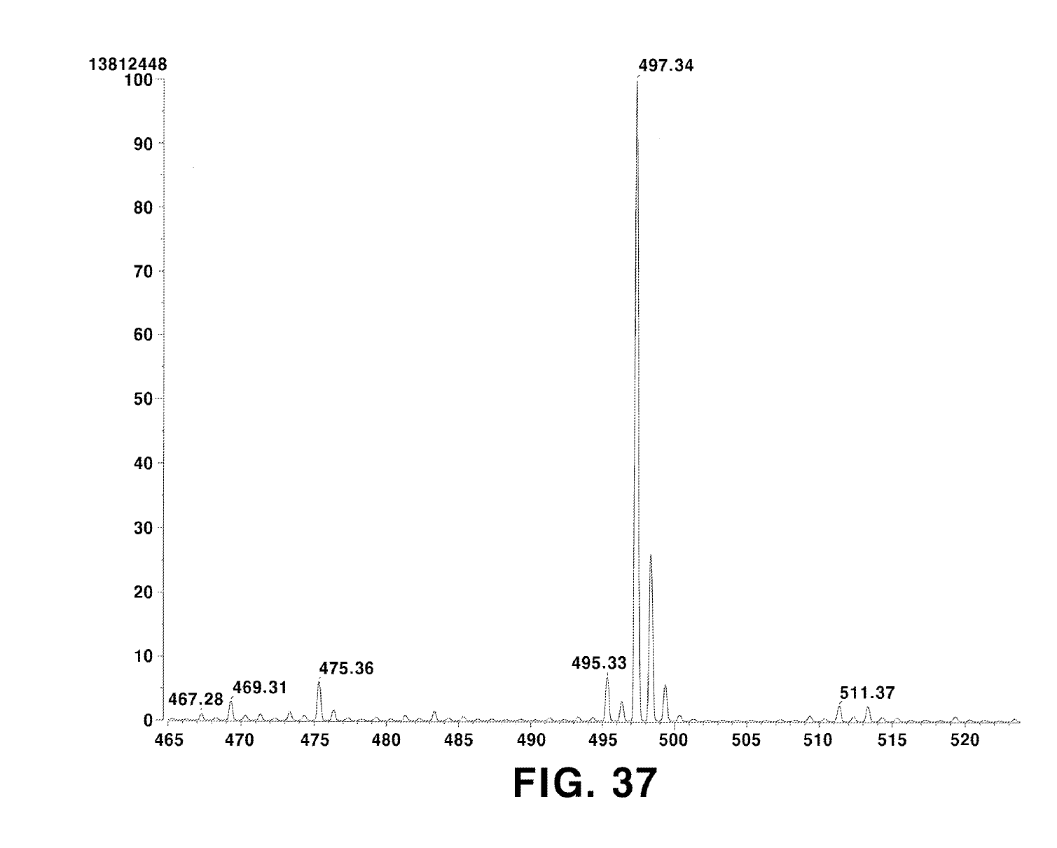

Finally, the chemical structure of the compound was confirmed by measuring its molecular weight and interpreting it with NMR. As shown in FIG. 37, [M+H]⁺ was observed at m/z 475, and [M+Na]⁺ was observed at m/z 497, which suggests that the compound of the present invention has a molecular weight of 474. In addition, a high-resolution ESI-mass spectrum was measured so as to confirm a molecular formula. As a result, [M+Na]⁺ was observed at m/z 497.2705, which complied with a molecular formula of C₂₄H₄₂O₉Na(Δ−2.1 mmu). These results exactly coincided with the chemical structure interpreted by NMR.

(5) Compound A57-4-gly-5

In order to investigate a chemical structure of compound A57-4-gly-5, It was dissolved in CDCl₃, and subjected to ¹H NMR, ¹³C NMR, ¹H-¹H COSY, HMQC and HMBC analyses.

Measurement and Interpretation of a ¹H NMR Spectrum:

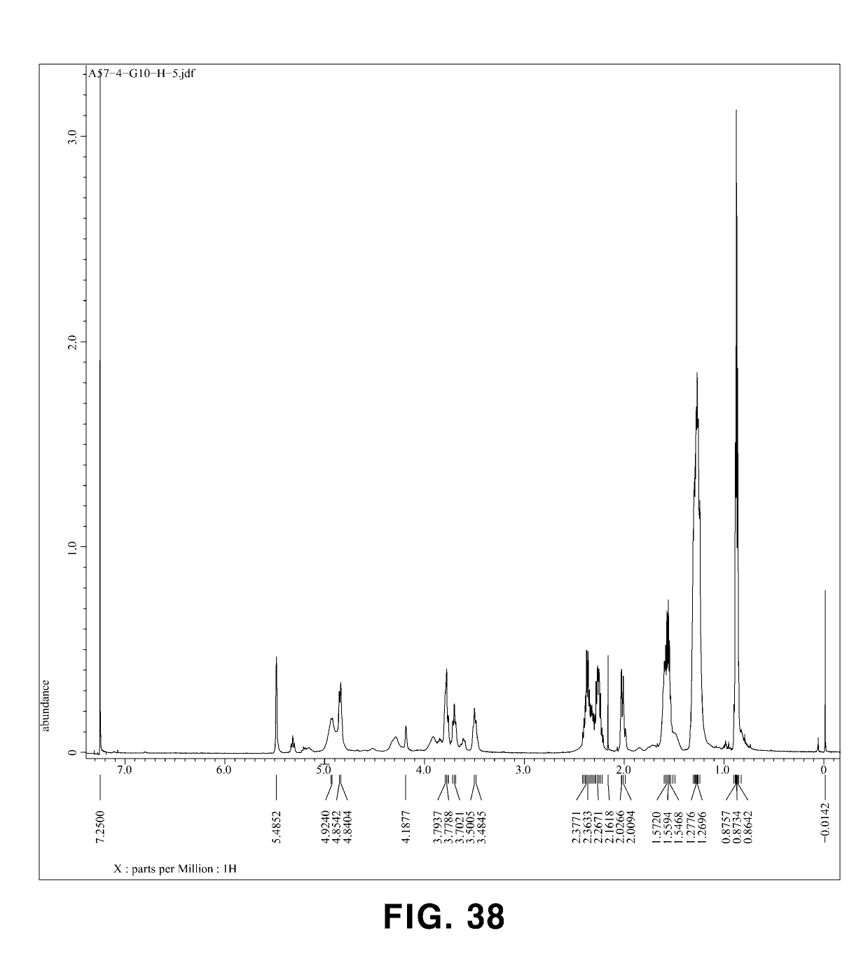

As a result of measuring a ¹H NMR spectrum by using CDCl₃ as a solvent (FIG. 38), six oxygenated methine protons at 5.49, 4.85, 3.80, 3.79, 3.70, and 3.50 ppm; eight methylene protons at 2.37, 2.26, 1.59, 1.57, 1.30, 1.25, and 1.2-1.4(×2) ppm; and two methyl protons at 0.88 and 0.87 ppm were observed. Also, four hydroxyl protons were observed at 4.93(×2), 4.29 and 3.92 ppm.

Measurement and Interpretation of a ¹³C NMR Spectrum:

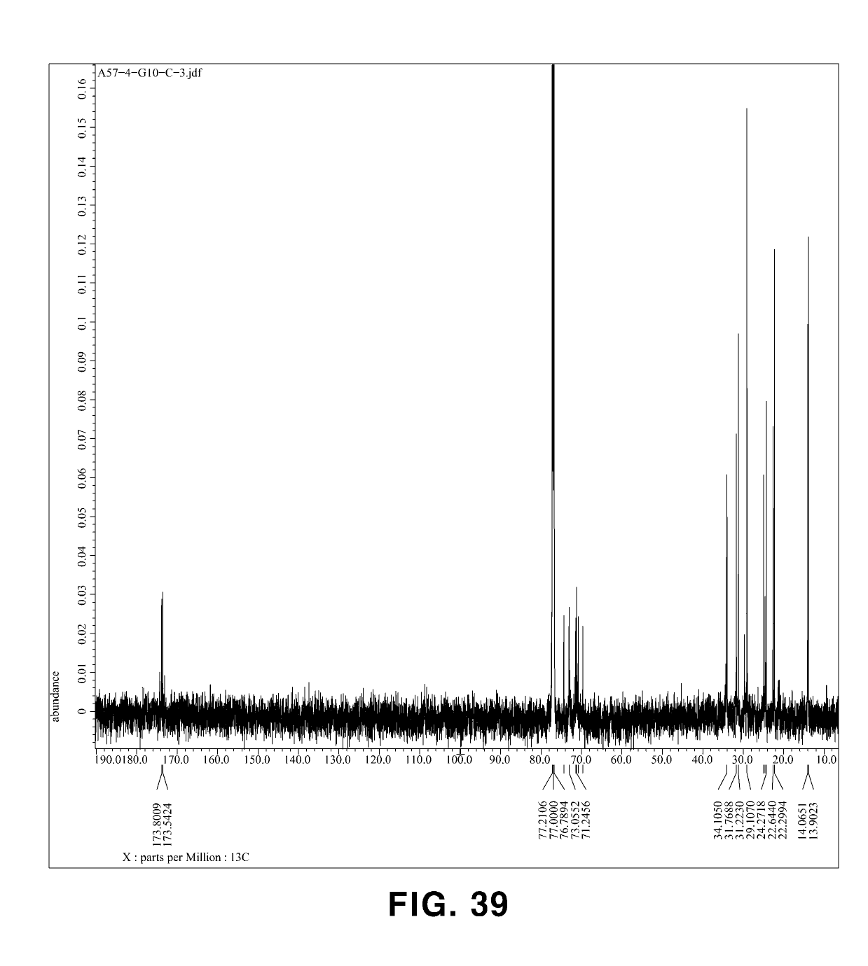

As a result of measuring a ¹³C NMR spectrum by using CDCl₃ as a solvent (FIG. 39), total eighteen peaks were observed. That is, two ester carbonyl carbons at 173.8 and 173.5 ppm; six oxygenated methine carbons at 74.4, 73.1, 71.4, 71.2, 70.8, and 69.7 ppm; eight methylene carbons at 34.1, 34.0, 31.2(×2), 24.6, 24.3, and 22.3(×2) ppm; and two methyl carbons at 14.1 and 13.9 ppm were observed.

Measurement and Interpretation of a ¹H-¹H COSY Spectrum:

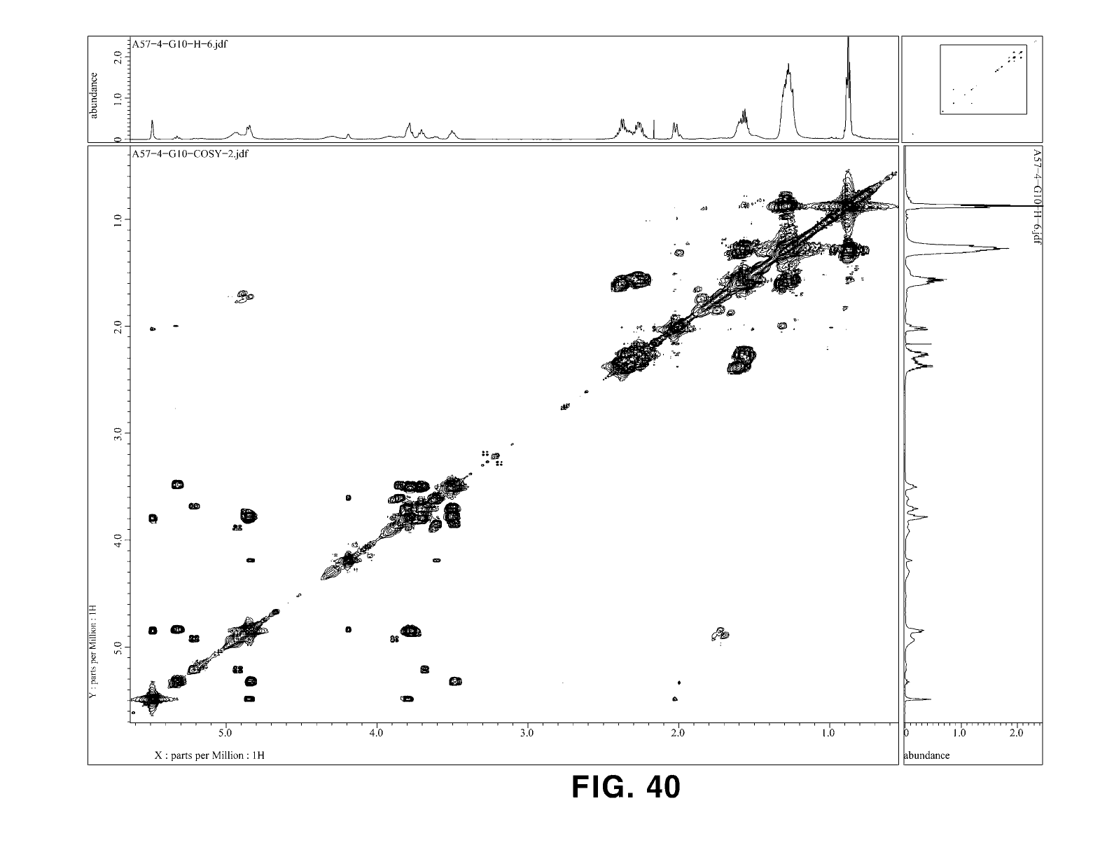

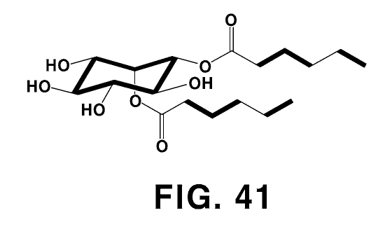

In order to investigate a chemical structure of compound A57-4-gly-5, a ¹H-¹H COSY spectrum which provides the information about correlations (³J_{H-H}) between coupled protons was measured and interpreted (FIG. 40). As a result, the correlation between oxygenated methine protons was observed, suggesting the presence of an inositol moiety. It has been found that excepting the proton at 5.49 ppm from coupling constants of the protons, the rest of protons occupy an axial position. From these results, the inositol moiety constituting the compound of the present invention was identified as a myo-inositol. Further, four partial structures present in an acyl chain were identified (FIG. 41).

Measurement and Interpretation of a HMQC Spectrum:

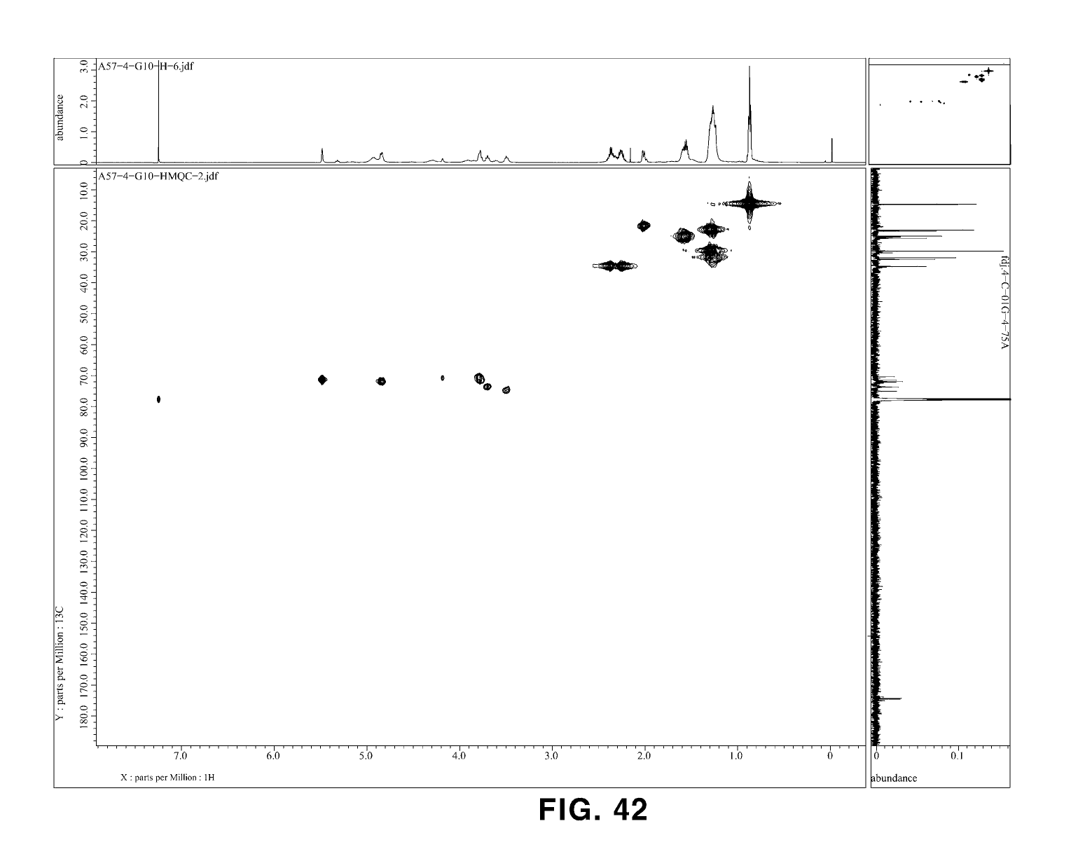

In order to investigate a chemical structure of compound A57-4-gly-5, a HMQC spectrum which provides the information about correlations (¹J_{C-H}) between hydrogen and carbon was measured and interpreted (FIG. 42). As a result, the correlation between all hydrogens and carbons constituting the compound of the present invention was identified.

Measurement and Interpretation of a HMBC Spectrum:

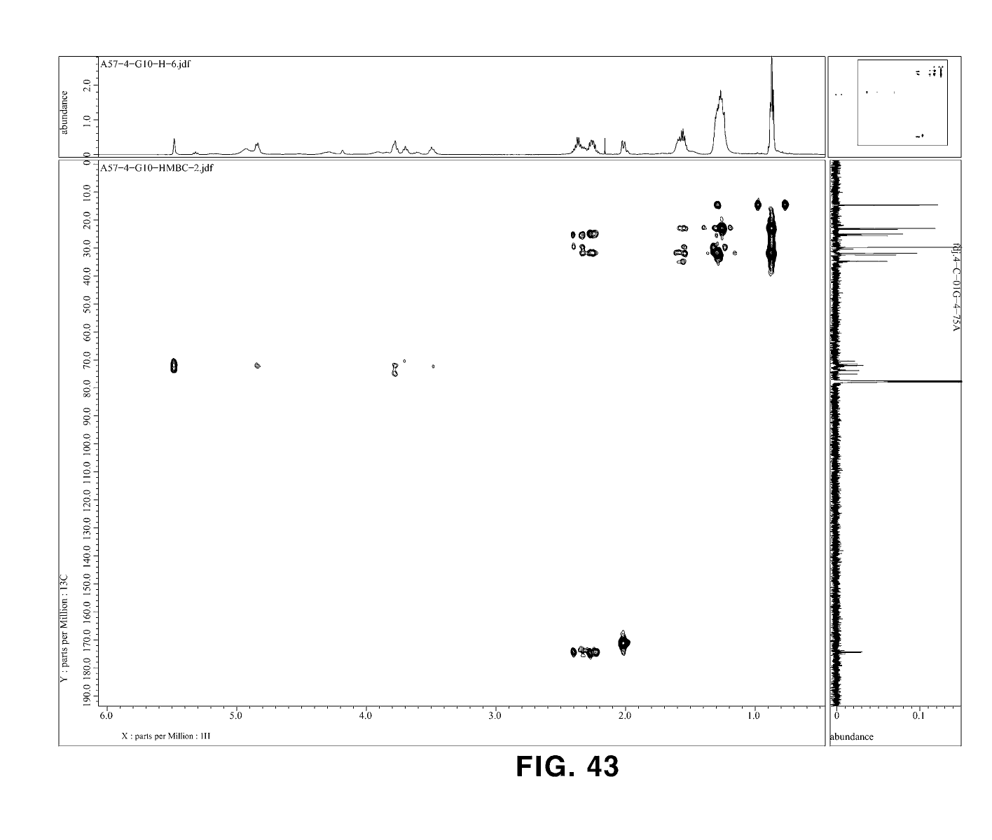

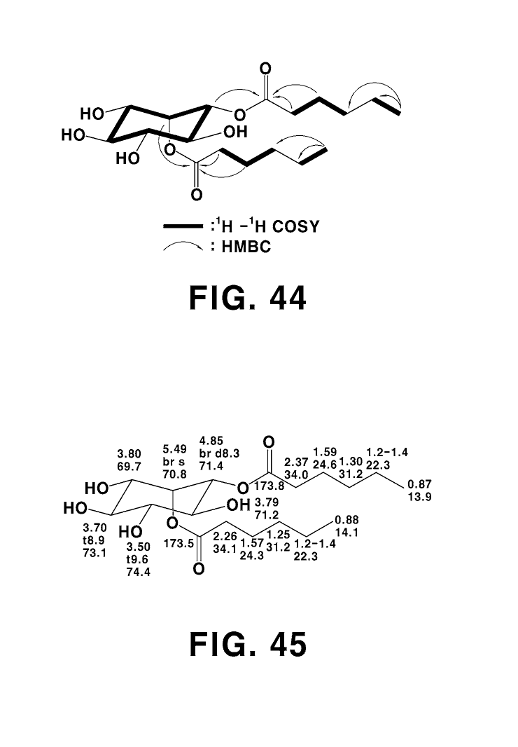

In order to investigate a chemical structure of compound A57-4-gly-5, a HMBC spectrum which provides the information about chemical shift (²J_{C-H}, ³J_{C-H}) of carbon atoms that are about 2-3 bonds away from the proton to which they correlate was measured and interpreted (FIG. 43). As a result, the long-range chemical shift correlation from methyl protons at 0.88, and 0.87 ppm to methylene carbon at 31.2 ppm, that from methylene protons at 2.37, and 1.59 ppm to ester carbonyl carbons at 2.26, and 1.57 ppm, and that from methylene protons at 2.26, and 1.57 ppm to ester carbonyl carbon at 173.5 ppm were observed, which suggests the presence of two hexanoyl groups. Also, the long-range chemical shift correlation from oxygenated methine protons at 5.49, 4.85 ppm to ester carbonyl carbons at 173.5, 173.8 ppm, respectively, was observed, which identifies the position to which two hexanoyl groups coupled. Therefore, it has been found that the compound of the present invention has a chemical structure where two hexanoyl groups coupled to the myo-inositol moiety. The chemical structure of the compound according to the present invention was determined as illustrated in FIG. 44, and the reversal of each proton and carbon peak was shown in FIG. 45. As a result of searching databases and articles based on the chemical structure as identified above, it has been found that the compound of the present invention is novel.

Measurement and Interpretation of an ESI-Mass Spectrum:

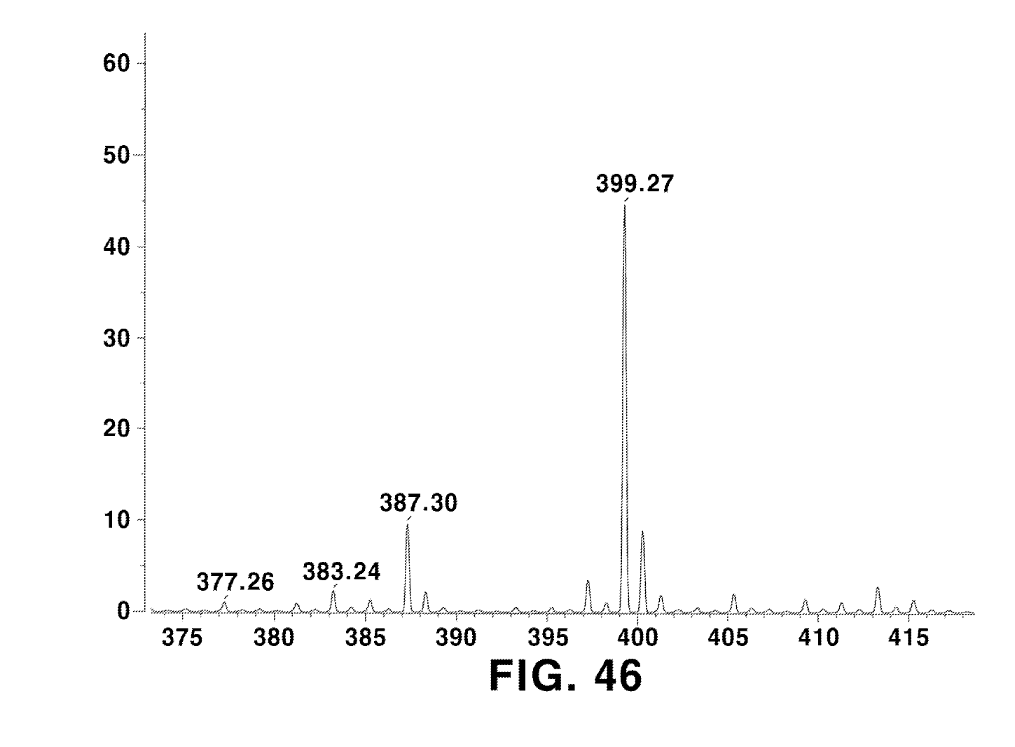

Finally, the chemical structure of the compound was confirmed by measuring its molecular weight and interpreting it with NMR. As shown in FIG. 46, [M+Na]⁺ was observed at m/z 399, which suggests that the compound of the present invention has a molecular weight of 376. In addition, a high-resolution ESI-mass spectrum was measured so as to confirm a molecular formula. As a result, [M+Na]⁺ was observed at m/z 399.1998, which complied with a molecular formula of C₁₈H₃₂O₈Na(Δ+0.3 mmu). These results exactly coincided with the chemical structure interpreted by NMR.

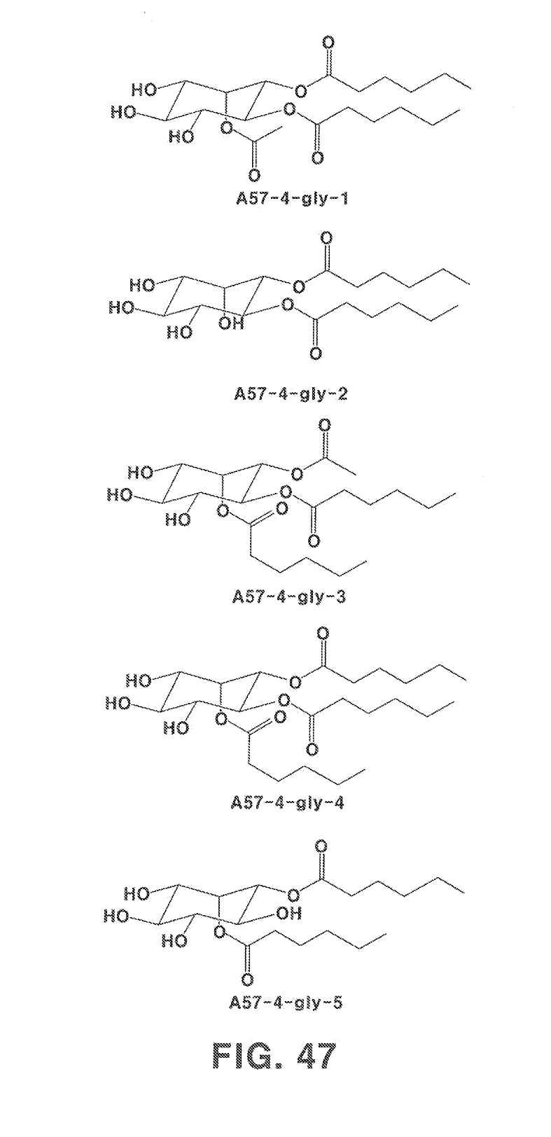

The chemical structure of the novel bio-surfactant material identified in the present invention is illustrated in FIG. 47.

Surface tension is increased in proportion to molecular interaction, and hydrocarbons or organic polymers show low surface tension due to their weak molecular interaction. Surface tension is represented by N/m. Since surfactants have hydrophobic and hydrophilic groups, when added to water, its surface tension is lowered. Surface tension of water, mercury and glycerin were measured and the results are shown in Table 1. As shown in Table 1, the higher the molecular interaction is, the greater the surface tension is. The compounds represented by

Formulae 2 to 6 according to the present invention showed a surface tension ranging from 22.40 to 28.71 N/m at 1.5 mg/L. Water as a control had a surface tension of 72.8 N/m. It has been found that since they exhibited significantly lower surface tension than water, mercury and glycerin, the compounds represented by Formulae 2 to 6 can be effectively used as a strong bio-surfactant.

Surface tension of compounds represented by Formulae 2 to 6

| No | Sample name | Surface Tension (N/m) |
|---|---|---|
| Formula 2 | A57-4-gly-1 | 22.90 |
| Formula 3 | A57-4-gly-2 | 22.40 |
| Formula 4 | A57-4-gly-3 | 28.71 |
| Formula 5 | A57-4-gly-4 | 25.28 |
| Formula 6 | A57-4-gly-5 | 22.44 |
| — | Water | 72.8 |
| — | Mercury | 486 |
| — | Glycerin | 63 |

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A compound represented by the Formula 1: 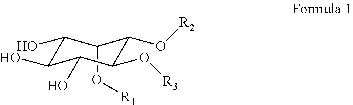
wherein R₁ to R₃ are independently hydrogen or —COR₄;
R₄ is C₁-C₁₀ alkyl; and
at least one of R₁ to R₃ is —COC₅H₁₁.

2. The compound of claim 1, wherein R₄ is C₁-C₈ alkyl.

3. The compound of claim 2, wherein R₄ is C₁-C₅ alkyl.

4. The compound of claim 3, wherein R₄ is methyl or pentyl.

5. The compound of claim 1, wherein the compound is selected from the group consisting of compounds represented by the following Formulae 2 to 6:

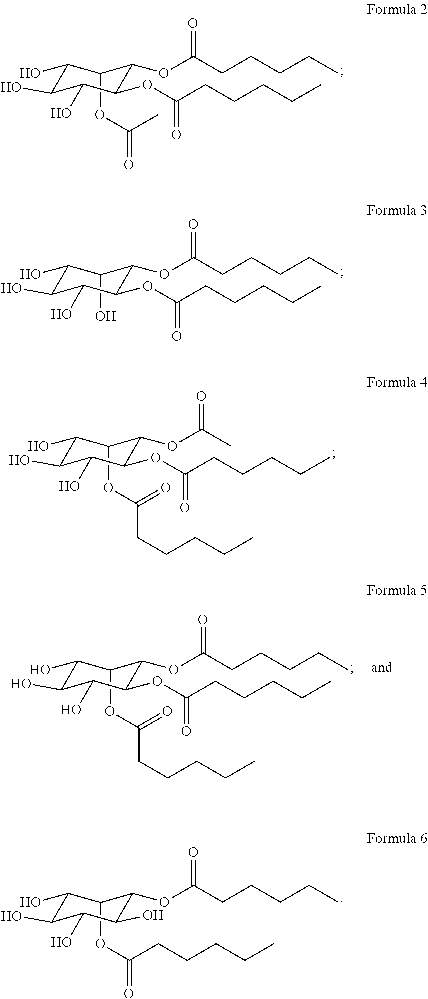 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]

6. The compound of claim 5, wherein the compound is isolated from a yeast strain.

7. The compound of claim 6, wherein the yeast strain is an Aureobasidium sp. strain deposited with Accession No. KCCM11373P.

8. The compound of claim 1, wherein the compound is a bio-surfactant.

9. A cleaning composition comprising the compound of claim 8.

10. A cosmetic composition comprising the compound of claim 8.

11. A method for preparing a bio-surfactant, comprising:
isolating a compound selected from Formulae 2 to 6: 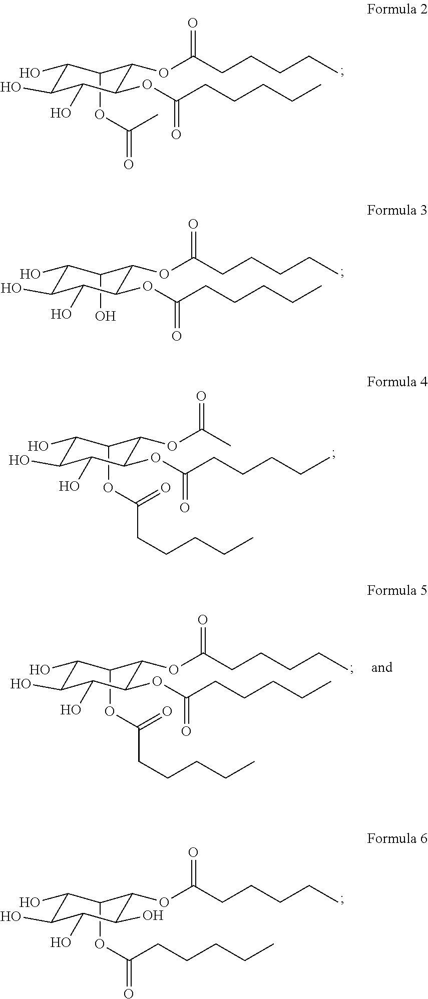 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]
wherein the compound is isolated from an Aureobasidium sp. strain deposited with Accession No. KCCM11373P.

12. An Aureobasidium sp. strain deposited with Accession No. KCCM11373P; wherein the strain produces a compound selected from Formulae 2 to 6: 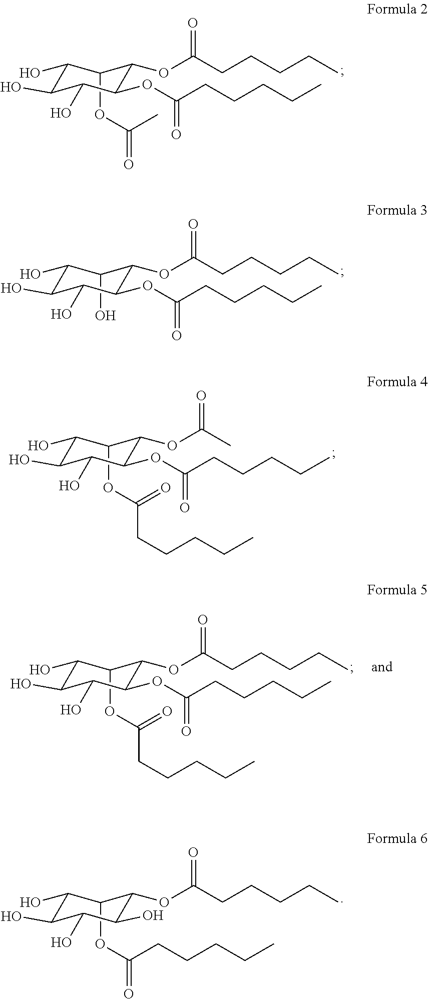 [SMILES: CCCCCC(=O)OC1C(O)C(O)C(O)C(OC(C)=O)C1OC(=O)CCCCC ; CCCCCC(=O)OC1C(O)C(O)C(O)C(O)C1OC(=O)CCCCC ; CCCCCC(=O)OC1C(O)C(O)C(O)C(OC(=O)CCCCC)C1OC(C)=O ; CCCCCC(=O)OC1C(O)C(O)C(O)C(OC(=O)CCCCC)C1OC(=O)CCCCC ; CCCCCC(=O)OC1C(O)C(O)C(O)C(O)C1OC(=O)CCCCC]
